(19) United States
(12) Patent Application Publication
(10) Pub. No.: US 2016/0245794 A1
(43) Pub. Date: Aug. 25, 2016

(54) METHODS AND COMPOSITIONS FOR SYSTEMIC LUPUS ERYTHEMATOSUS

(71) Applicant: Nodality, Inc., South San Francisco, CA (US)

(72) Inventors: Rachael Hawtin, San Carlos, CA (US); Wouter Korver, Mountain View, CA (US); Erik Evensen, Foster City, CA (US)

(21) Appl. No.: 14/837,902

(22) Filed: Aug. 27, 2015

Related U.S. Application Data
(60) Provisional application No. 62/042,733, filed on Aug. 27, 2014.
(60) Provisional application No. 62/079,189, filed on Nov. 13, 2014.

(51) Int. Cl. G01N 33/50

(52) U.S. Cl. CPC G01N 33/505; G01N 33/5052; G01N 33/5047

(57) ABSTRACT

The invention provides methods and compositions for the diagnosis, prognosis, and/or treatment response characterization of individuals suffering from systemic lupus erythematosus (SLE) using single cell network profiling.

### CROSS-REFERENCE

This application claims the benefit of U.S. Provisional Application No. 62/042,733, filed Aug. 27, 2014 [Attorney Docket No. 33118-767.101], and U.S. Provisional Application No. 62/079,189, filed Nov. 13, 2014 [Attorney Docket No. 33118-767.102], which applications are incorporated herein by reference.

### BACKGROUND OF THE INVENTION

Systemic Lupus Erythematosus (SLE) is a chronic multisystem autoimmune disorder with a broad spectrum of clinical presentations encompassing many organs and tissues. Its highly variable clinical course is characterized by periods with minimal or absent disease activity interspersed with periods of active disease (flare), with the potential to ultimately result in organ-related damage, due to both disease and treatment. To date, there are no reliable indices that allow stratification of patients into subgroups whose diagnosis, prognosis and/or treatment response characteristics can be predicted.

### SUMMARY OF THE INVENTION

In one aspect the invention provides methods. In certain embodiments, the invention provides a method of determining the status of an individual diagnosed with or suspected of having SLE comprising (i) determining the activation level of an activatable element in a cell from a sample from the individual; and (ii) based on the level determined in (i), determining the status of the individual. In certain embodiment, the individual has been diagnosed with SLE and the status is current status of the disease, likelihood of a future status of the disease, or likelihood of response to treatment. The cell can be treated with a modulator, such as CD40L, CpG-C, Anti-IgD, IL-1β, LPS, Pam3CSK4, PMA, R848, IFNα, IFNγ, IL-2, IL-4, IL-6, IL-7, IL-10, IL-15, IL-21, IL-27, or GMCSF. In certain embodiments, the activatable element is p-Akt, p-CREB, p-Erk, IkB, p-c-Jun, p-P38, p-S6, p-Stat3, p-Stat1, p-Stat3, p-Stat5, or p-Stat6. In certain embodiments, the cell is a T cell, a B cell, or a monocyte, or a subset selected from the group in TABLE1. In certain embodiments, the activation level of two activatable elements is determined and the determination of the status comprises finding a ratio of the levels of the two activatable elements, for example, in the cell treated with a modulator.

In certain embodiments the invention provides a method of screening an agent for potential use as a therapeutic agent in SLE, comprising exposing cells to the agent and determining the activation level of one or more activatable elements single cells, and determining the suitability of the agent for potential use as a therapeutic agent based on the activation level determined. In certain embodiments, the single cells are treated with a modulator, such as CD40L, CpG-C, Anti-IgD, IL-113, LPS, Pam3CSK4, PMA, R848, IFNα, IFNγ, IL-2, IL-4, IL-6, IL-7, IL-10, IL-15, IL-21, IL-27, or GMCSF. In certain embodiments, the activatable element is p-Akt, p-CREB, p-Erk, IkB, p-c-Jun, p-P38, p-S6, p-Stat3, p-Stat1, p-Stat3, p-Stat5, or p-Stat6. In certain embodiments, the cell in which the activation level of the activatable element is determined is a T cell, a B cell, or a monocyte, or a subset selected from the group in the TABLE 1. In certain embodiments, the activation level of two activatable elements is determined and the determination of the suitability of the agent comprises finding a ratio of the levels of the two activatable elements, such as wherein the cell is treated with a modulator.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows that IFN modulated signaling was more heterogeneous in SLE patients than in healthy controls. Healthy (top), SLE (middle), SLE overlaid on Healthy, showing greater heterogeneity in SLE samples.

FIG. 2 shows that a subgroup of SLE patient samples signaled lower for IFNa and higher for IFNg. Other SLE samples signaled like healthy. Nodes displayed are IFNα→p-STAT5 and IFNγ→p-STAT1 in B cells as indicated. A. lower box on left to upper box on right, Interferon Group, with low IFNa and high IFNg signaling; upper box on right and lower box on left: SLE patients who behave like healthy, i.e., high IFNa and low IFNg. B. Healthy compared to SLE

FIG. 3 shows results for the SLE-IFN subgroup showing differences from SLE patients not in the subgroup. Higher TLR 7/8 modulated signaling was observed in B cells and dendritic cells but not in monocytes; lower TLR9 signaling was observed in B cells, and lower TLR1/2 and TLR4 modulated signaling was observed in monocytes.

FIG. 4 shows enhanced p-STAT-1 and reduced p-STAT3 signaling was observed upon cytokine modulation in the IFN subgroup.

FIG. 5 shows signaling nodes interrogated in comparison of PBMCs of SLE patients and healthy donors.

FIG. 6 shows modulated signaling more heterogenous in SLE compared to HD.

FIG. 7 shows basal p-ERK levels not different between HD and SLE (unmodulated signaling is not elevated in SLE), PMA→p-ERK not different between SLE and HD (signaling capacity in SLE B cells is intact), and CD40L→p-ERK is reduced in SLE compared to HD.

FIG. 8 shows signaling pathway specific effects of belimumab treatment, including reduced CD40L signaling in samples from patients treated with belimumab, TLR (CpG-B) modulated signaling is the same in patients with or without belimumab.

FIG. 9 shows that B cell subset numbers are reduced in a subset of SLE patients; belimumab treatment reduced overall numbers in treated patients (B) compared to untreated (NB) or healthy donors (HD) (arrow in third column from left, SLE B), primarily due to lower numbers of naïve CD27-IgD+ B cells (arrow in fourth column from right). T cell and monocyte numbers were similar between HD and SLE (not shown).

FIG. 10 shows clustering based on signaling stratifies SLE patients beyond clinical factors. Patient subgroups were identified using K-means clustering with log 2Fold modulated signaling data referenced to the healthy range of signaling. Data is presented as a parallel plot with lines representing each cluster showing the median signal on each node.

FIG. 11 shows lower TLR 7/8/9 modulated B cell signaling with anti-malarial drug treatment.

### DETAILED DESCRIPTION OF THE INVENTION

The present invention incorporates information disclosed in other applications and texts. The following patent and other publications are hereby incorporated by reference in their entireties: Haskell et al, Cancer Treatment, 5th Ed., W.B. Saunders and Co., 2001; Alberts et al., The Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and 1B, Growth Factors, 1996. Other conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y.; and Sambrook, Fritsche and Maniatis. “Molecular Cloning A laboratory Manual” 3rd Ed. Cold Spring Harbor Press (2001), all of which are herein incorporated in their entirety by reference for all purposes.

Also, patents and applications that are incorporated by reference include U.S. Pat. Nos. 7,381,535, 7,393,656, 7,563,584, 7,695,924, 7,695,926, 7,939,278, 8,148,094, 8,187,885, 8,198,037, 8,206,939, 8,214,157, 8,227,202, 8,242,248; U.S. patent application Ser. Nos. 11/338,957, 11/655,789, 12/061,565, 12/125,759, 12/125,763, 12/229,476, 12/432,239, 12/432,720, 12/471,158, 12/501,274, 12/501,295, 12/538,643, 12/551,333, 12/581,536, 12/606,869, 12/617,438, 12/687,873, 12/688,851, 12/703,741, 12/713,165, 12/730,170, 12/778,847, 12/784,478, 12/877,998, 12/910,769, 13/082,306, 13/091,971, 13/094,731, 13/094,735, 13/094,737, 13/098,902, 13/098,923, 13/098,932, 13/098,939, 13/384,181; International Applications Nos. PCT/US2011/001565, PCT/US2011/065675, PCT/US2011/026117, PCT/US2011/029845, PCT/US2011/048332; and U.S. Provisional Applications Ser. Nos. 60/304,434, 60/310,141, 60/646,757, 60/787,908, 60/957,160, 61/048,657, 61/048,886, 61/048,920, 61/055,362, 61/079,537, 61/079,551, 61/079,579, 61/079,766, 61/085,789, 61/087,555, 61/104,666, 61/106,462, 61/108,803, 61/113,823, 61/120,320, 61/144,68, 61/144,955, 61/146,276, 61/151,387, 61/153,627, 61/155,373, 61/156,754, 61/157,900, 61/162,598, 61/162,673, 61/170,348, 61/176,420, 61/177,935, 61/181,211, 61/182,518, 61/182,638, 61/186,619, 61/216,825, 61/218,718, 61/226,878, 61/236,281, 61/240,193, 61/240,613, 61/241,773, 61/245,000, 61/254,131, 61/263,281, 61/265,585, 61/265,743, 61/306,665, 61/306,872, 61/307,829, 61/317,187, 61/327,347, 61/350,864, 61/353,155, 61/373,199, 61/374,613, 61/381,067, 61/382,793, 61/423,918, 61/436,534, 61/440,523, 61/469,812, 61/499,127, 61/515,660, 61/521,221, 61/542,910, 61/557,831, 61/558,343, 61/565,391, 61/565,929, 61/565,935, 61/591,122, 61/640,794, 61/658,092, 61/664,426, and 61/693,429.

The status of an individual may be associated with a diagnosis, prognosis, choice or modification of treatment, and/or monitoring of a disease, disorder, or condition. Through the determination of the status of an individual, a health care practitioner can assess whether the individual is in the normal range for a particular condition or whether the individual has a pre-pathological or pathological condition warranting monitoring and/or treatment. Thus, in some embodiments, the status of an individual involves the classification, diagnosis, prognosis of a condition or outcome after administering a therapeutic to treat the condition.

The subject invention also provides kits (described in detail below in the section entitled “Kits”) for use in determining the status of an individual, the kit comprising one or more specific binding elements for activatable elements, optionally surface markers, and may additionally comprise one or more therapeutic agents. These binding elements can also be called “stains” which can include an antibody and a label. The kit may further comprise a software package for data analysis of the different populations of cells, which may include reference profiles for comparison with the test profile.

The discussion below describes some of the preferred embodiments with respect to particular diseases. However, it should be appreciated that the principles may be useful for the analysis of many other diseases as well.

### INTRODUCTION

SLE is a chronic multisystem autoimmune disorder with a broad spectrum of clinical presentations encompassing many organs and tissues. Its highly variable clinical course is characterized by periods with minimal or absent disease activity interspersed with periods of active disease (flare), with the potential to ultimately result in organ-related damage, due to both disease and treatment.

Classification criteria for SLE were developed in 1971, revised in 1982, and revised again in 1997 by The American College of Rheumatology (ACR). One of any possible constellations of 4 of 11 criteria must be met for a classification of SLE, underscoring its clinical heterogeneity. The ACR criteria were developed and validated in patients with established disease and, therefore, may not capture patients who have early or limited disease; conversely, other individuals will meet criteria but may not have SLE. This clinical heterogeneity has been an important obstacle for the development of drugs and diagnostics for SLE.

Given the lack of access of many patients to rheumatologic evaluation, disease heterogeneity both in clinical features and as a result of its changing manifestations over time, as well as challenges in diagnosing early or limited disease, accurate incidence data are difficult to obtain. Estimated worldwide incidence rates of SLE range from approximately 1 to 10 per 100,000 person-years and prevalence rates generally range from 20 to 70 per 100,000. SLE is primarily a disease of reproductive age women though it can occur at any age in both genders. In the United States, there is an increased risk among reproductive age African Americans; however, in other populations, the highest age-specific incidence rates occur in women after age 40. SLE is two to four times more frequent and more severe among nonwhite populations around the world and tends to be more severe in male, pediatric, and late-onset cases.

A number of factors are thought to contribute to the development and manifestations of SLE, including genetic influences, epigenetic regulation of gene expression, environmental exposures, female hormones and gender, and aberrant immune cell function.

In order to facilitate clinical studies and clinical decision-making, several disease activity indices have been developed and validated in the evaluation of patients with SLE. Each has strengths and limitations, and no currently available index is uniformly adept in describing all SLE clinical features with respect to activity, damage, responsiveness to treatment, and reversibility. The SLEDAI (SLE Disease Activity Index) is a list of 24 items, 16 of which are clinical and 8 of which are laboratory results, scored based on the presence or absence of manifestations within the previous 10 days, with organ involvement being weighted. The final score can range from 0 to 105. Scores >20 are rare, and a score ≧6 constitutes active disease generally requiring therapy. The SLEDAI was modified in the Safety of Estrogens in Lupus Erythematosus National Assessment (SELENA) trial by clarifying some of the definitions of activity but not changing the scoring system. While the SELENA-SLEDAI is a well-accepted measurement of disease activity, this composite score has several limitations that confound accurate assessment of patients. A number of other indices have been designed in an attempt to better monitor disease activity, but there remains room for improvement and consensus among clinical investigators has not been achieved.

Each of the indices includes various (objective) laboratory parameters, along with historical and clinical findings, but the stratification of patients into subgroups whose prognosis and treatment response characteristics can be predicted remains impossible at present. This also limits the utility of the available instruments in clinical trials, to select a homogeneous group of patients with respect to prognosis, disease stage or severity, likelihood to respond to specific interventions, etc. The application of flow cytometry in SLE has to date been limited to research purposes and has focused largely on the enumeration of individual peripheral blood (PB) cell subsets based on the expression of cell surface markers. By contrast, SCNP examines the functional status of a variety of cell subsets present in the PBMC population. SCNP also allows the assessment of responsiveness by any of a variety of cell (sub)populations to modulators and/or drugs, thus providing a view of the integration of genetic and epigenetic features that differ from patient to patient and in association with disease status and demographic characteristics.

Treatment approaches for SLE are varied and have historically involved symptomatic management, hormonal manipulation, and immunotherapies. Decisions regarding treatment choice are generally made on empirical grounds and clinical experience, and significant morbidity results from treatment as well as from disease. New approaches, such as biologic therapies and small molecule drugs, are being developed to correct aberrant immune-cell function, with the hope that they will have greater efficacy and improved tolerability over current options. Despite the active research in SLE, morbidity and mortality remain significant in this generally young population, with a 10-year survival rate of approximately 70%. Thus, there remains a need for the development of improved diagnostic, disease activity and treatment response monitoring tools, as well as effective therapeutics.

The invention provides methods and compositions related to SLE by assessing the levels of one or more activatable elements in cells of an individual. The cells may be exposed to a modulator. In certain embodiments, single cells are assessed. In certain embodiments, the cells are assessed by flow cytometry. In certain embodiments, the cells are assessed by mass spectrometry. The information regarding the activatable elements may be combined with other information about the individual, such as race, age, gender, medication use and/or duration, duration of disease, previous disease status, anti-dsDNA antibody status, interferon status, ANA, anemia, proteinuria, complement, anti-SM, and any other suitable characteristic.

In one aspect, the invention provides methods and compositions for determining the status of an individual diagnosed with SLE. The status may be any status pertinent to the monitoring, treatment, or other aspect of SLE in the individual. The status may be present disease status. The status may be predicted future status, such as predicting the probability of a flare at a certain future time, for example, likelihood of an increase of more than 3 in the SLEDAI score at a certain time point, such as 1, 2, 3, 4, 5, 6, 9, or 12 months from the time the sample was taken. The status may be likelihood of response to treatment, e.g., response to belimumab. The status may be membership in a certain strata of patient stratification, e.g., for disease severity, progression, likelihood of response to treatment, likelihood of future flare, etc. In certain embodiments, the invention provides a method of treatment of an individual suffering from SLE comprising treating the patient with a treatment based on predicting flare by any method as described herein.

In certain embodiments, flare is predicted by determining an activation level of a STAT, such as pSTAT5, in cells, such as B cells, from an individual that have been modulated with an interferon, such as interferon alpha; and an activation level of a different STAT, such as pSTAT1, in cells, such as B cells, from an individual that have been modulated with a different interferon, such as interferon gamma. A ratio of the two levels may be taken. In certain embodiments, flare is predicted by determining an activation level of a STAT, such as pSTAT1, in cells, such as B cells, monocytes, or T cells, e.g., monocytes, from an individual that have been modulated with cytokine, such IL-6, IL-10, IL-21, or IL-27, e.g., IL-10; and an activation level of a STAT, such as a different STAT, e.g. pSTAT3, in cells, such as B cells, monocytes, or T cells, e.g., monocytes, from an individual that have been modulated with the same cytokine, such IL-6, IL-10, IL-21, or IL-27, e.g., IL-10. A ratio of the two levels may be taken.

In another aspect, the invention provides methods of screening for agents that may be useful in the treatment of SLE.

In general, in methods of the invention, cells from a sample from an individual, e.g., a blood sample or PBMC sample, are assessed for the levels of an activatable element by use of a detectable state-binding element that binds to molecules of the activatable element in a particular activation state and detection of the binding element, as described below for SCNP. In some cases the cells may be exposed to a modulator before assessment of the activatable element(s). Any suitable activatable element may be used; in certain embodiments, the activatable element is activated by phosphorylation or cleavage. Any suitable detectable binding element may be used; in certain embodiments, the binding element comprises an antibody, e.g. a labeled antibody. In certain embodiments, the label comprises a fluorescent label. In certain embodiments, the label comprises a mass tag. Any suitable detection method may be used. In certain embodiments, detection is by flow cytometry. In certain embodiments, detection is by mass spectrometry.

The methods may further include gating cells so that only cells of one or more populations are included in analysis. One method of gating gates cells for health, e.g., by scatter, Amine Aqua binding, and/or by measuring levels of an indicator of apoptosis, such as cPARP levels. Cells may be gated by population. In certain embodiments, one or more populations as shown below are used:

| T Cells | B Cells | Monocytes | NK enriched |
|---|---|---|---|
| CD4− T Cells | CD27− IgD− | mDC | CD3−CD20−CD14− |
| | B Cells | | |
| CD4+ T Cells | CD27− IgD+ | pDC | |
| | B Cells | | |
| CD45RA− CD4− | CD27+ IgD− | | |
| T Cells | B Cells | | |
| CD45RA− CD4+ | CD27+ IgD+ | | |
| T Cells | B Cells | | |
| CD45RA+ CD4− | | | |
| T Cells | | | |
| CD45RA+ CD4+ | | | |
| T Cells | | | |

Suitable activatable elements for use in the invention are any activatable elements as described herein. In certain embodiments, the activatable element is one or more of p-Akt, p-CREB, p-Erk, IkB, p-c-Jun, p-P38, p-S6, p-Stat3, p-Stat1, p-Stat3, p-Stat5, or p-Stat6. In certain embodiments, the activatable elements comprise p-S6, p-ErK, or p-Stat1, or any combination thereof. In certain embodiments, the activatable element comprises pS6. In certain embodiments, the activatable elements comprise p-S6 and p-Erk. In certain embodiments, the activatable elements comprise p-S6 and p-Stat1. In certain embodiments, the activatable elements comprise p-S6, p-Erk, and p-Stat1.

Suitable modulators for use in the invention are any modulators as described herein. In certain embodiments, the modulator(s) is one or more of CD40L, CpG-C, Anti-IgD, IL-1β, LPS, Pam3CSK4, PMA, R848, IFNα, IFNγ, IL-2, IL-4, IL-6, IL-7, IL-10, IL-15, IL-21, IL-27, or GMCSF. In certain embodiments, IFNg is used. In certain embodiments, a TLR modulator is used, such as a modulator of TLR7/8, e.g. R848, or TLR1/2, e.g., PAM3CSK4 or TLR4, e.g., LPS, or TLR9, e.g., CpG-B, CpG-C.

In certain embodiments, a particular modulator→readout (node), optionally in a specific cell subset, may be used. In certain embodiments, one or more of IFNa→p-Stat5, e.g., in B cells; IFNa→p-Stat1, e.g., in B cells; IFNa→p-Stat3, e.g., in B cells; IFNg→p-Stat1, e.g., in B cells; IFNg→p-Stat3, e.g., in B cells; IFNg→p-Stat5, e.g., in B cells; TLR7/8 (TLR modifier such as R848)→p-Erk, e.g, in B cells; TLR7/8→IkB, e.g., in B cells and/or pDCs; Pam3CSK4 (TLR1/2), LPS (TLR4)→p-Erk in monocytes; TLR1/2 (e.g. PAM3CSK4)→one or more of p38, iKB, p-c-Jun, or pERK, e.g., in monocytes, may be used. In certain embodiments, one or more of IFNa→p-Stat5 in B cells, and IFNg→p-Stat1 in B cells is used. In certain embodiments, one or more of IFNa→p-Stat5 in B cells, T cells, and/or monocytes and IFNg→p-Stat1 in B cells, T cells, and/or monocytes is used. In certain embodiments, IFNα-→p-Stat5; IFNγ→p-Stat1; TLR7/8→p-Erk; TLR7/8→iKB in B cells is used. In certain embodiments, CD40L→IkB in B cells is used. In certain embodiments, TLR9→pErk; TLR9→pP38; TLR9→IkB in B cell subsets is used. In certain embodiments, TLR1/2→p-P38; TLR1/2→IkB; TLR1/2→p-pErk in monocytes is used. In certain embodiments, IL-10→p-Stat1; IL-10→p-Stat5 in T cells and/or monocytes is used. In certain embodiments, IFNα, IFNγ, IL-6, IL-10, IL-21, IL-27→p-Stat1, -3, in T cells, B cells, and/or monocytes is used. In certain embodiments, IL-2→p-Stat5; IL-4→p-Stat6 in T cells is used. In certain embodiments IL-4→p-Stat5 in B cells is used. In certain embodiments, IL-6→p-Stat5; IL-6→p-Stat3 in T cells or monocytes is used. In certain embodiments, IL-7→p-Stat5 in T cells or B cells is used. In certain embodiments, IL-10→p-Stat3; IL-10→p-Stat5 in B cells is used. In certain embodiments, IL-21→p-Stat3; IL-21→p-Stat5 in T cells and/or B cells is used. In certain embodiments, IL-27→p-Stat1; IL-27→p-Stat5 in T cells is used. In certain embodiments, Anti-IgD→p-Akt, p-S6 in B cells is used. In certain embodiments, IL-1β→p-Erk in monocytes is used. In certain embodiments, TLR7/8→IkB in monocytes is used.

In certain embodiments, e.g., methods and compositions for patient stratification in clinical trials, such as trials in which a particular treatment, e.g. drug or combination of drugs, is tested for SLE, readouts comprise one or more of p-Stat1 p-Stat3, p-Stat5, or p-Stat6. In certain embodiments, readouts comprise at least p-Stat1. Other useful readouts include one or more of p-p38, p-Erk, p-S6, and/or IkB Modulators may include one or more of IL-4, IL-6, IL-7, IFNg, IFNa, IL-27, IL-2, IL-10, IL-21, CD40L, CpG Type C, Pam3CSK4, PMA, IgD, R848, IL-1b, CpG Type B. Nodes for use in these embodiments may include at least 1, 2, 3, 4, 5, 6, 7, or 8 of IL-6→p-Stat1 (for example, in CD4+ T cells); IFNg→p-Stat1 (e.g., in B cells); IL-27→p-Stat5 (e.g., in CD45RA+CD4− Tcells); IFNa2→p-Stat5 (e.g., in CD4+ T cells); IL-2→p-Stat5 (e.g., in CD45RA-CD4− T cells); IL-10→p-Stat3 (e.g., in CD3-CD20-CD14− cells); IL-7→p-Stat5 (e.g. in CD4− T cells); IL-4→p-Stat6 (e.g., in CD45RA-CD4− T cells); LPS→p-Erk (e.g., in monocytes); Pam3CSK4→p-p38 (e.g., in monocytes); R848→IkB (e.g., in monocytes); PMA→p-p38 (e.g., in monocytes); CD40L→IkB (e.g., in B cells); CpG Type C→p-Erk (e.g., in B cells); CpG Type B→p-Erk (e.g., in CD27−IgD+ Bcells). In certain embodiments, the node or nodes comprises IL-6→p-Stat1 (for example, in CD4+ T cells). In certain embodiments, nodes include at least 1, 2, 3, 4, 5, 6, 7 or 8 of IL-4→p-Stat6 (e.g., in monocytes); IL-6→p-Stat1 (e.g., in T cells); IFNg→p-Stat1 (e.g., in B cells), IFNa→p-Stat1 (e.g., in T cells); IL-27→p-Stat1 (e.g., in T cells); IL-2→p-Stat5 (e.g., in T cells), IL-10→p-Stat3 (e.g., in monocytes); IL-21→p-Stat3 (e.g., in B cells); CD40L→p-S6 (e.g., in B cells); CpG Type C→p-S6 (e.g., in B cells); Pam3CSK4→p-Erk (e.g., in monocytes); PMA→p-Erk (e.g., in monocytes); IgD→p-S6 (e.g., in B cells); R848→p-Erk (e.g., in B cells); IL-1b p-Erk (e.g., in monocytes); CpG Type B→IkB (e.g., in B cells); LPS→p-Erk (e.g., in monocytes). In addition, the invention provides compositions comprising the necessary detectable binding elements for detecting any of the activatable elements described in this paragraph, such as 1, 2, 3, 4, 5, 6, 7, or 8 detectable binding elements (e.g., antibodies) for detecting 1, 2, 3, 4, 5, 6, 7, or 8 of p-Stat1, p-Stat3, p-Stat5, p-Stat6, p-Erk, p-S6, IkB, p-p38. Detectable binding elements may also include binding elements specific to one or more cell surface markers for classifying cells into the populations listed in this paragraph The compositions may comprise modulators, such as 1, 2, 3, 4, 5, 6, 7, 8, or more than 8 of IL-4, IL-6, IL-7, IFNg, IFNa, IL-27, IL-2, IL-10, IL-21, CD40L, CpG Type C, Pam3CSK4, PMA, IgD, R848, IL-1b, CpG Type B. In certain embodiments the invention provides an assay template, e.g., a multiwall plate such as one or more 96-well microtiter plates, in whose wells are provided the necessary modulators for one or more of the nodes listed herein, e.g., at least 1, 2, 3, 4, 5, 6, 7 or 8 wells provided with the necessary modulators for at least 1, 2, 3, 4, 5, 6, 7 or 8 of IL-4→p-Stat6 (e.g., in monocytes); IL-6→p-Stat1 (e.g., in T cells); IFNg→p-Stat1 (e.g., in B cells), IFNa→p-Stat1 (e.g., in T cells); IL-27→p-Stat1 (e.g., in T cells); IL-2→p-Stat5 (e.g., in T cells), IL-10→p-Stat3 (e.g., in monocytes); IL-21→p-Stat3 (e.g., in B cells); CD40L→p-S6 (e.g., in B cells); CpG Type C→→p-S6 (e.g., in B cells); Pam3CSK4→p-Erk (e.g., in monocytes); PMA→p-Erk (e.g., in monocytes); IgD→p-S6 (e.g., in B cells); R848→p-Erk (e.g., in B cells); IL-1b p-Erk (e.g., in monocytes); CpG Type B→IkB (e.g., in B cells); LPS→p-Erk (e.g., in monocytes). In certain embodiments the invention provides an assay template, e.g., a multiwall plate such as one or more 96-well microtiter plates, in whose wells are provided the necessary detectable binding elements, e.g., antibodies for one or more of the nodes listed herein, e.g., at least 1, 2, 3, 4, 5, 6, 7 or 8 wells provided with the necessary detectable binding elements, e.g., antibodies, for at least 1, 2, 3, 4, 5, 6, 7 or 8 of IL-4→p-Stat6 (e.g., in monocytes); IL-6p-Stat1 (e.g., in T cells); IFNg→p-Stat1 (e.g., in B cells), IFNa→p-Stat1 (e.g., in T cells); IL-27→p-Stat1 (e.g., in T cells); IL-2→p-Stat5 (e.g., in T cells), IL-10→p-Stat3 (e.g., in monocytes); IL-21→p-Stat3 (e.g., in B cells); CD40L→p-S6 (e.g., in B cells); CpG Type C→p-S6 (e.g., in B cells); Pam3CSK4→p-Erk (e.g., in monocytes); PMA→p-Erk (e.g., in monocytes); IgD→p-S6 (e.g., in B cells); R848→p-Erk (e.g., in B cells); IL-1→b p-Erk (e.g., in monocytes); CpG Type B→IkB (e.g., in B cells); LPS→p-Erk (e.g., in monocytes).

In certain embodiments, combinations of nodes, such as ratios are used. In certain embodiments, the ratio of IFNa→p-Stat5, e.g., in B cells and IFNg→p-Stat1, e.g., in B cells, is used. In certain embodiments, a ratio is used of two of p-Stat1, p-Stat3, or p-Stat5 response (e.g., p-Stat1 and p-Stat3, or p-Stat3 and p-Stat5, or p-Stat1 and p-Stat5), where the modulator may be one of the modulators described herein, for example IFNa, IFNg, IL-6, IL-10, IL-21, or IL-27 in some cases in a cell subset as described herein, for example, B cells, T cells, or monocytes.

The nodes, singly or in combination, may be used to evaluate the status of the individual, for example, present disease status or future disease status (e.g., likelihood of flare, for example, likelihood of an increase of more than 1, or more than 2, or more than 3 in the SLEDAI score at a certain time point, such as 1, 2, 3, 4, 5, 6, 9, or 12 months from the time the sample was taken), or likelihood of response to treatment. The nodes, singly or in combination, may also be used as markers to screen candidate agents as potential drugs for treatment; e.g., a change in the node or nodes in response to exposure to a candidate agent can indicate that the agent has potential as a treatment for SLE. For example, a TLR (e.g., TLR9)→p-ERK node, for example, in B cells. In certain embodiments, the individual is treated for a predicted flare based on the above method, for example, by administration of an agent known to be effective in treating SLE. The treatment may be given at a time that is optimal or near optimal for preventing or ameliorating the predicted flare.

The invention also provides kits for determining the status of an individual, for example, an individual suffering from SLE, such as kits for prediction of flare in SLE, wherein the kit contains one or more detectable binding elements for detection of one or more of the activatable described herein; one or more modulators for modulating cells from the individual, as described herein; one or more detectable binding elements for determining one or more surface markers to classify cells from the individual, as described herein; instructions for use, either provided with the other components of the kit or accessible specifically for use with the components (e.g., electronically accessible); reagents for determining cell viability, e.g., Amine Aqua; one or more detectable binding elements for determining cell health, e.g., detectable binding element to cPARP; and/or suitable packaging for the one or more components of the kit, such as packaging suitable to allow the kit to be transported from a supplier to an user in one or more packages, such as in 1, 2, 3, 4, 5, 6, 7, or 8 packages. For example a kit may contain at least one, or at least 2, or at least 3, or at least 4 of a detectable binding element for detecting p-Akt, p-CREB, p-Erk, IkB, p-c-Jun, p-P38, p-S6, p-Stat3, p-Stat1, p-Stat3, p-Stat5, or p-Stat6. A kit may contain at least one, or at least 2, or at least 3, or at least 4 of a modulator selected from the group consisting of CD40L, CpG-C, Anti-IgD, IL-1β, LPS, Pam3CSK4, PMA, R848, IFNα, IFNγ, IL-2, IL-4, IL-6, IL-7, IL-10, IL-15, IL-21, IL-27, or GMCSF. A kit may contain at least one, or at least 2, or at least 3, or at least 4 of a detectable binding element for detecting cell surface markers to classify cells as members of a cells set or subset, such as the sets and subsets shown in Table 1 or Table 3; such cell surface markers are well-known in the art and include without limitation CD3, CD4, CD45RA, CD27, CD19, CD20, CD14, CD25, CD33, CD69, and Foxp3. Kits may further include reagents, buffers, hardware, software (including software provided electronically or as a tangible medium) and/or other materials useful in performing the assays for which the components of the kit are used.

### Single Cell Network Profiling (SCNP)

Single cell network profiling (SCNP) is a method that can be used to analyze activatable elements, such as phosphorylation sites of proteins, in signaling pathways in single cells in response to modulation by signaling agonists or inhibitors (e.g., kinase inhibitors). Other examples of activatable elements include an acetylation site, a ubiquitination site, a methylation site, a hydroxylation site, a SUMOylation site, or a cleavage site. Activation of an activatable element can involve a change in cellular localization or conformation state of individual proteins, or change in ion levels, oxidation state, pH etc. It is useful to classify cells and to provide diagnosis or prognosis as well as other activities, such as drug screening or research, based on the cell classifications. SCNP is one method that can be used in conjunction with an analysis of cell health, but there are other methods that may benefit from this analysis. Embodiments of SCNP are shown in references cited herein. See for example, U.S. Pat. No. 7,695,924, U.S. patent application Ser. No. 13/580,660, and U.S. Patent Application No. 61/729,171, all of which are hereby incorporated by reference in their entirety. Other exemplary previously filed patent applications have elements that may be used in the present process and compositions and include the use of control beads, the use of monitoring software, and the use of automation. See U.S. Ser. Nos. 12/776,349, 12/501,274 and 12/606,869 respectively. All applications are hereby incorporated by reference in their entireties. See also U.S. Ser. No. 61/557,831 which is hereby incorporated by reference.

In general, the invention involves the detection of the level of a form of an activatable element, for example, an activated form, in single cells (the “activation level” of the activatable element). In some cases, the forms, e.g., activated forms, of a plurality of activatable elements are detected. The cells may be exposed to one or more modulators before the detection of the activatable element. Detection may be achieved by any suitable method known in the art; in some cases, a detectable binding element is bound to the form, e.g., activated form, of the activated element and detected. Activatable elements, modulators, binding elements, detection, and methods of analysis of data are described below.

### Samples and Sampling

The invention involves analysis of cells from one or more cell populations, where the cell populations are derived from one or more samples removed from an individual or individuals. An individual or a patient is any multi-cellular organism; in some embodiments, the individual is an animal, e.g., a mammal. In some embodiments, the individual is a human. In all cases, the cell population is derived from a sample that has been removed from the individual and placed in an environment in which it is no longer in contact with, and interacting with, the body as a whole, and any cells and cell populations involved in events in the culture are thus removed from interactions with cells, tissues, and organs of the body, and any factors produced by the cells, tissues, and organs, that would normally and naturally occur in a natural, i.e., whole-body, setting.

The sample may be any suitable type that allows for the derivation of cells from one or more cell populations. Samples may be obtained once or multiple times from an individual. Multiple samples may be obtained from different locations in the individual (e.g., blood samples, bone marrow samples and/or lymph node samples), at different times from the individual (e.g., a series of samples taken to monitor response to treatment or to monitor for return of a pathological condition), or any combination thereof. These and other possible sampling combinations based on the sample type, location and time of sampling allows for the detection of the presence of pre-pathological or pathological cells, the measurement treatment response and also the monitoring for disease.

When samples are obtained as a series, e.g., a series of blood samples, the samples may be obtained at fixed intervals, at intervals determined by the status of the most recent sample or samples or by other characteristics of the individual, or some combination thereof. For example, samples may be obtained at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for sampling and the availability of sampling facilities, thus approximate intervals corresponding to an intended interval scheme are encompassed by the invention. As an example, an individual who has undergone treatment for a rheumatoid arthritis may be sampled (e.g., by blood draw) relatively frequently (e.g., every month or every three months) to determine the effect of the treatment and whether or not treatment should be modified.

Generally, the most easily obtained samples are fluid samples. Fluid samples include normal and pathologic bodily fluids and aspirates of those fluids. Fluid samples also comprise rinses of organs and cavities (lavage and perfusions). Bodily fluids include whole blood, samples derived from whole blood such as peripheral blood mononuclear cells (PBMCs), bone marrow aspirate, synovial fluid, cerebrospinal fluid, saliva, sweat, tears, semen, sputum, mucus, menstrual blood, breast milk, urine, lymphatic fluid, amniotic fluid, placental fluid and effusions such as cardiac effusion, joint effusion, pleural effusion, and peritoneal cavity effusion (ascites). Rinses can be obtained from numerous organs, body cavities, passage ways, ducts and glands. Sites that can be rinsed include lungs (bronchial lavage), stomach (gastric lavage), gastrointestinal track (gastrointestinal lavage), colon (colonic lavage), vagina, bladder (bladder irrigation), breast duct (ductal lavage), oral, nasal, sinus cavities, and peritoneal cavity (peritoneal cavity perfusion).

In certain embodiments the sample from which cells from one or more cell populations are derived is blood. The blood may be untreated or minimally treated, beyond having been removed from the natural and more complex milieu of the body of the individual. In certain embodiments, the sample is treated by methods well-known in the art to contain only, or substantially only, PBMC.

In certain embodiments, the sample is a synovial fluid sample. In certain embodiments, combinations of blood or blood-derived samples (e.g. PBMC samples) and synovial fluid samples are used.

Solid tissue samples may also be used, either alone or in conjunction with fluid samples. Solid samples may be derived from individuals by any method known in the art including surgical specimens, biopsies, and tissue scrapings, including cheek scrapings. Surgical specimens include samples obtained during exploratory, cosmetic, reconstructive, or therapeutic surgery. Biopsy specimens can be obtained through numerous methods including bite, brush, cone, core, cytological, aspiration, endoscopic, excisional, exploratory, fine needle aspiration, incisional, percutaneous, punch, stereotactic, and surface biopsy.

Certain fluid samples can be analyzed in their native state, though isolated and removed from the natural milieu of the whole body, with or without the addition of a diluent or buffer. Alternatively, fluid samples may be further processed to obtain enriched or purified discrete cell populations prior to analysis. Numerous enrichment and purification methodologies for bodily fluids are known in the art. A common method to separate cells from plasma in whole blood is through centrifugation using heparinized tubes. By incorporating a density gradient, further separation of the lymphocytes from the red blood cells can be achieved. A variety of density gradient media are known in the art including sucrose, dextran, bovine serum albumin (BSA), FICOLL diatrizoate (Pharmacia), FICOLL metrizoate (Nycomed), PERCOLL (Pharmacia), metrizamide, and heavy salts such as cesium chloride. Alternatively, red blood cells can be removed through lysis with an agent such as ammonium chloride prior to centrifugation.

Whole blood can also be applied to filters that are engineered to contain pore sizes that select for the desired cell type or class. For example, rare pathogenic cells can be filtered out of diluted, whole blood following the lysis of red blood cells by using filters with pore sizes between 5 to 10 μm, as disclosed in U.S. patent application Ser. No. 09/790,673. Alternatively, whole blood can be separated into its constituent cells based on size, shape, deformability or surface receptors or surface antigens by the use of a microfluidic device as disclosed in U.S. patent application Ser. No. 10/529,453.

Select cell populations can also be enriched for or isolated from whole blood through positive or negative selection based on the binding of antibodies or other entities that recognize cell surface or cytoplasmic constituents. For example, U.S. Pat. No. 6,190,870 to Schmitz et al. discloses the enrichment of tumor cells from peripheral blood by magnetic sorting of tumor cells that are magnetically labeled with antibodies directed to tissue specific antigens.

Solid tissue samples may require the disruption of the extracellular matrix or tissue stroma and the release of single cells for analysis. Various techniques are known in the art including enzymatic and mechanical degradation employed separately or in combination. An example of enzymatic dissociation using collagenase and protease can be found in Wolters G H J et al. An analysis of the role of collagenase and protease in the enzymatic dissociation of the rat pancrease for islet isolation. Diabetologia 35:735-742, 1992. Examples of mechanical dissociation can be found in Singh, N P. Technical Note: A rapid method for the preparation of single-cell suspensions from solid tissues. Cytometry 31:229-232 (1998). Alternately, single cells may be removed from solid tissue through microdissection including laser capture microdissection as disclosed in Laser Capture Microdissection, Emmert-Buck, M. R. et al. Science, 274(8):998-1001, 1996.

The cells can be separated from body samples by centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells may be obtained. Alternatively, a heterogeneous cell population can be used. Cells can also be separated by using filters. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Ser. Nos. 12/432,720 and 13/493,857 and U.S. Pat. No. 8,227,202. See also, the commercial products from companies such as BD and BCI. See also U.S. Pat. Nos. 7,381,535 and 7,393,656.

In some embodiments, the cells are cultured post collection in a media suitable for revealing the activation level of an activatable element (e.g. RPMI, DMEM) in the presence, or absence, of serum such as fetal bovine serum, bovine serum, human serum, porcine serum, horse serum, or goat serum. When serum is present in the media it could be present at a level ranging from 0.0001% to 30%.

### Modulators

In some embodiments, the methods and composition utilize a modulator. A modulator can be an activator, an inhibitor or a compound capable of impacting a cellular pathway. Modulators can also take the form of environmental cues and inputs.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, cells are exposed to at least 2, 3, 4, 5, 6, 7, 8, 9, or 10 modulators.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments any suitable amount of serum is used. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

In some embodiments, modulators produce different activation states depending on the concentration of the modulator, duration of exposure or whether they are used in combination or sequentially with other modulators.

In some embodiments the modulator is selected from the group consisting of growth factor, cytokine, adhesion molecule modulator, drugs, hormone, small molecule, polynucleotide, antibodies, natural compounds, lactones, chemotherapeutic agents, immune modulator, carbohydrate, proteases, ions, reactive oxygen species, peptides, and protein fragments, either alone or in the context of cells, cells themselves, viruses, and biological and non-biological complexes (e.g. beads, plates, viral envelopes, antigen presentation molecules such as major histocompatibility complex). In some embodiments, the modulator is a physical stimuli such as heat, cold, UV radiation, and radiation.

In some embodiments, the modulator is an activator. In some embodiments the modulator is an inhibitor. In some embodiments, cells are exposed to one or more modulators. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the modulator is a B cell receptor modulator. In some embodiments, the B cell receptor modulator is a B cell receptor activator. An example of B cell receptor activator is a cross-linker of the B cell receptor complex or the B-cell co-receptor complex. In some embodiments, cross-linker is an antibody or molecular binding entity. In some embodiments, the cross-linker is an antibody. In some embodiments, the antibody is a multivalent antibody. In some embodiments, the antibody is a monovalent, bivalent, or multivalent antibody made more multivalent by attachment to a solid surface or tethered on a nanoparticle surface to increase the local valency of the epitope binding domain.

In some embodiments, the cross-linker is a molecular binding entity. In some embodiments, the molecular binding entity acts upon or binds the B cell receptor complex via carbohydrates or an epitope in the complex. In some embodiments, the molecular is a monovalent, bivalent, or multivalent is made more multivalent by attachment to a solid surface or tethered on a nanoparticle surface to increase the local valency of the epitope binding domain.

In some embodiments, the cross-linking of the B cell receptor complex or the B-cell co-receptor complex comprises binding of an antibody or molecular binding entity to the cell and then causing its crosslinking via interaction of the cell with a solid surface that causes crosslinking of the BCR complex via antibody or molecular binding entity.

In some embodiments, the crosslinker is F(ab)₂ IgM, IgG, IgD, polyclonal BCR antibodies, monoclonal BCR antibodies, Fc receptor derived binding elements and/or a combination thereof. The Ig can be derived from a species selected from the group consisting of mouse, goat, rabbit, pig, rat, horse, cow, shark, chicken, or llama. In some embodiments, the crosslinker is F(ab)₂ IgM, Polyclonal IgM antibodies, Monoclonal IgM antibodies, Biotinylated F(ab)2 IgG/M, Biotinylated Polyclonal IgM antibodies, Biotinylated Monoclonal IgM antibodies and/or combination thereof.

In some embodiments, the inhibitor is an inhibitor of a cellular factor or a plurality of factors that participates in a cellular pathway (e.g. signaling cascade) in the cell. In some embodiments, the inhibitor is a kinase or phosphatase inhibitor. Examples of kinase inhibitors are recited above.

In certain embodiments in which the status of an individual with rheumatoid arthritis is categorized, the modulator is one or more of anti-CD3 antibody, Fab2IgM, IFNα2, IL-6, IL-10, LPS, IgD, R848, or TNFα or any combination thereof

In certain embodiments in which an individual is treated based on the status of one or more activatable elements in response to modulation, the modulator is one or more of of anti-CD3 antibody, Fab2IgM, IFNα2, IL-6, IL-10, and TNFα, or any combination thereof. In certain of these embodiments, the modulator is one or more of IL-6, IFNa, or TNFα.

### Activatable Elements

An “activatable element,” as that term is used herein, is an element that exists in at least two states that are distinct and that are distinguishable. The activation state of an individual activatable element is either in the on or off state. An activatable element is generally a part of a cellular protein or other constituent. In some cases the term “activatable element” is used synonymously with the term “protein or constituent with an activatable element,” which is clear from context. As an illustrative example, and without intending to be limited to any theory, an individual phosphorylatable site on a protein will either be phosphorylated and then be in the “on” state or it will not be phosphorylated and hence, it will be in the “off” state. See Blume-Jensen and Hunter, Nature, vol 411, 17 May 2001, p 355-365. The terms “on” and “off,” when applied to an activatable element that is a part of a cellular constituent, are used here to describe the state of the activatable element (e.g., phosphorylated is “on” and non-phosphorylated is “off”), and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is typically measured through the use of a binding element that recognizes a specific activation state, only those activatable elements in the specific activation state recognized by the binding element, representing some fraction of the total number of activatable elements, will be bound by the binding element to generate a measurable signal.

The measurable signal corresponding to the summation of individual activatable elements of a particular type that are activated in a single cell is the “activation level” for that activatable element in that cell.

At the next level of data aggregation, activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

In some embodiments, the basis determining the activation levels of one or more activatable elements in cells may use the distribution of activation levels for one or more specific activatable elements which will differ among different conditions. A certain activation level, or more typically a range of activation levels for one or more activatable elements seen in a cell or a population of cells, is indicative that that cell or population of cells belongs to a certain condition. Other measurements, such as cellular levels (e.g., expression levels) of biomolecules that may not contain activatable elements, may also be used to determine the activation state data of a cell in addition to activation levels of activatable elements; it will be appreciated that these levels also will follow a distribution, similar to activatable elements. Thus, the activation level or levels of one or more activatable elements, alternatively or in addition, with levels of one or more of biomolecules that may not contain activatable elements, of one or more cells in a discrete population of cells may be used to determine the activation state data of the discrete cell population.

In some embodiments, the basis for determining the activation state data of a discrete cell population may use the position of a cell in a contour or density plot. The contour or density plot represents the number of cells that share a characteristic such as the activation level of activatable proteins in response to a modulator. For example, when referring to activation levels of activatable elements in response to one or more modulators, normal individuals and patients with a condition might show populations with increased activation levels in response to the one or more modulators. However, the number of cells that have a specific activation level (e.g. specific amount of an activatable element) might be different between normal individuals and patients with a condition. Thus, the activation state data of a cell can be determined according to its location within a given region in the contour or density plot.

### Additional Elements

Instead of, or in addition to activation levels of intracellular activatable elements, expression levels of intracellular or extracellular biomolecules, e.g., proteins may be used alone or in combination with activation states of activatable elements when evaluating cells in a cell population. Further, additional cellular elements, e.g., biomolecules or molecular complexes such as RNA, DNA, carbohydrates, metabolites, and the like, may be used instead of, or in addition to activatable states, expression levels or any combination of activatable states and expression levels in the determination of the physiological status of a population of cells encompassed here.

In some embodiments, other characteristics that affect the status of a cellular constituent may also be used to determine the activation state data of a discrete cell population. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Additional elements may also be used to determine the activation state data of a discrete cell population, such as the expression level of extracellular or intracellular markers, nuclear antigens, enzymatic activity, protein expression and localization, cell cycle analysis, chromosomal analysis, cell volume, and morphological characteristics like granularity and size of nucleus or other distinguishing characteristics. For example, T cells can be further subdivided based on the expression of cell surface markers such as CD4, CD45RA, CD27, and the like.

Alternatively, populations of cells can be aggregated based upon shared characteristics that may include inclusion in one or more additional cell populations or the presence of extracellular or intracellular markers, similar gene expression profile, nuclear antigens, enzymatic activity, protein expression and localization, cell cycle analysis, chromosomal analysis, cell volume, and morphological characteristics like granularity and size of nucleus or other distinguishing characteristics.

In some embodiments, the activation state data of one or more cells is determined by examining and profiling the activation level of one or more activatable elements in a cellular pathway.

Thus, the activation level of one or more activatable elements in single cells in a cell population from the sample is determined. Cellular constituents that may include activatable elements include without limitation proteins, carbohydrates, lipids, nucleic acids and metabolites. In some cases, the constituent is itself referred to as the “activatable element,” which is clear from context. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. The term “plurality” as used herein refers to two or more. In some embodiments, the activation levels of at least 2, 3, 4, 5, 6, 7, 8, 9, 10, or more than 10 intracellular activatable elements are determined in single cells of a discrete cell population. The activation levels may be determined in the same cell, or different cells of the same population.

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

In certain embodiments, the activatable element is an element that undergoes phosphorylation or dephosphorylation, or an element that undergoes cleavage.

In some embodiments, the activatable element is a protein. Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporate here by reference. See U.S. Ser. Nos. 12/432,720 and 13/493,857 and U.S. Pat. No. 8,227,202 and Shulz et al, Current Protocols in Immunology 2007, 7:8.17.1-20.

In some embodiments, the protein that may be activated is selected from the group consisting of HER receptors, PDGF receptors, FLT3 receptor, Kit receptor, FGF receptors, Eph receptors, Trk receptors, IGF receptors, Insulin receptor, Met receptor, Ret, VEGF receptors, erythropoetin receptor, thromobopoetin receptor, CD114, CD116, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk6, Cdk2, Cdk1, Cdk7, Cyclin D, Cyclin E, Cyclin A, Cyclin B, Rb, p16, pl4Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFκB), CREB, NFAT, ATF-2, AFT, Myc, Fos, Spl, Egr-1, T-bet, HIFs, FOXOs, E2Fs, SRFs, TCFs, Egr-1, β-catenin, FOXO STAT1, STAT 3, STAT 4, STAT 5, STAT 6, p53, WT-1, HMGA, pS6, 4EPB-1, eIF4E-binding protein, RNA polymerase, initiation factors, elongation factors. In one embodiment, the activatable element is a phosphorylated protein such as p-IkB, p-Akt, p-S6, p-NFκB proteins, p-IkK a/b, p-p38, p-Lck, P-Zap70, p-SRC Y418, p-Syk, or p-Erk 1/2.

In certain embodiments in which the status of an individual with rheumatoid arthritis is categorized, the activatable element is one or more of p-CD3ζ, p-Lck, p-Plcg2, p-ZAP70/Syk, p-STAT 1, p-STAT3, p-STAT5, p-Akt, p-P38, and p-S6, or any combination thereof. In certain of these embodiments, the activatable element is one or more of p-STAT1, p-STAT3, p-STAT4, or p-STAT 5, or any combination thereof

In certain embodiments in which an individual is treated based on the status of one or more activatable elements, the activatable element is one or more of p-Plcg2, p-CD3ζ, p-Lck, p-STAT1, p-STAT3, p-STAT4, p-STAT5, or IκBα, or any combination thereof. In certain of these embodiments, the activatable element is one or more of p-STAT1 or p-STAT3.

### Binding Elements

In some embodiments of the invention, the activation level of an activatable element is determined. One embodiment makes this determination by contacting a cell from a cell population with a binding element that is specific for an activation state of the activatable element. The term “Binding element” includes any molecule, e.g., peptide, nucleic acid, small organic molecule which is capable of detecting an activation state of an activatable element over another activation state of the activatable element. Binding elements and labels for binding elements are shown in U.S. Ser. Nos. 12/432,720 and 13/493,857 and U.S. Pat. No. 8,227,202 and the other applications incorporated above.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein include both naturally occurring and synthetic amino acids. For example, homo-phenylalanine, citrulline and noreleucine are considered amino acids for the purposes of the invention. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U138 Part 2 Aug. 22, 1999, both of which are expressly incorporated by reference herein.

Methods of the present invention may be used to detect any particular activatable element in a sample that is antigenically detectable and antigenically distinguishable from other activatable element which is present in the sample. For example, the activation state-specific antibodies of the present invention can be used in the present methods to identify distinct signaling cascades of a subset or subpopulation of complex cell populations; and the ordering of protein activation (e.g., kinase activation) in potential signaling hierarchies. Hence, in some embodiments the expression and phosphorylation of one or more polypeptides are detected and quantified using methods of the present invention. In some embodiments, the expression and phosphorylation of one or more polypeptides are detected and quantified using methods of the present invention. As used herein, the term “activation state-specific antibody” or “activation state antibody” or grammatical equivalents thereof, refer to an antibody that specifically binds to a corresponding and specific antigen. Preferably, the corresponding and specific antigen is a specific form of an activatable element. Also preferably, the binding of the activation state-specific antibody is indicative of a specific activation state of a specific activatable element.

In some embodiments, the binding element is an antibody. In some embodiment, the binding element is an activation state-specific antibody.

The term “antibody” includes full length antibodies and antibody fragments, and may refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and posses other variations. See U.S. Ser. Nos. 12/432,720 and 13/493,857 and U.S. Pat. No. 8,227,202 for more information about antibodies as binding elements.

Activation state specific antibodies can be used to detect kinase activity, however additional means for determining kinase activation are provided by the present invention. For example, substrates that are specifically recognized by protein kinases and phosphorylated thereby are known. Antibodies that specifically bind to such phosphorylated substrates but do not bind to such non-phosphorylated substrates (phospho-substrate antibodies) may be used to determine the presence of activated kinase in a sample.

The antigenicity of an activated isoform of an activatable element is distinguishable from the antigenicity of non-activated isoform of an activatable element or from the antigenicity of an isoform of a different activation state. In some embodiments, an activated isoform of an element possesses an epitope that is absent in a non-activated isoform of an element, or vice versa. In some embodiments, this difference is due to covalent addition of moieties to an element, such as phosphate moieties, or due to a structural change in an element, as through protein cleavage, or due to an otherwise induced conformational change in an element which causes the element to present the same sequence in an antigenically distinguishable way. In some embodiments, such a conformational change causes an activated isoform of an element to present at least one epitope that is not present in a non-activated isoform, or to not present at least one epitope that is presented by a non-activated isoform of the element. In some embodiments, the epitopes for the distinguishing antibodies are centered around the active site of the element, although as is known in the art, conformational changes in one area of an element may cause alterations in different areas of the element as well.

Many antibodies, many of which are commercially available (for example, see the websites of Cell Signaling Technology or Becton Dickinson) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, FLT3 receptor, Kit receptor, FGF receptors, Eph receptors, Trk receptors, IGF receptors, Insulin receptor, Met receptor, Ret, VEGF receptors, TIE1, TIE2, erythropoetin receptor, thromobopoetin receptor, CD114, CD116, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, cell cycle regulators, Cdk4, Cdk 6, Cdk 2, Cdk1, Cdk 7, Cyclin D, Cyclin E, Cyclin A, Cyclin B, Rb, p16, pl4Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets, Elk, SMADs, Rel-A (p65-NFκB), CREB, NFAT, ATF-2, AFT, Myc, Fos, Spl, Egr-1, T-bet, β-catenin, HIFs, FOXOs, E2Fs, SRFs, TCFs, Egr-1, β-FOXO STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, elongation factors. See also the proteins listed in the Examples below.

In some embodiments, an epitope-recognizing fragment of an activation state antibody rather than the whole antibody is used. In some embodiments, the epitope-recognizing fragment is immobilized. In some embodiments, the antibody light chain that recognizes an epitope is used. A recombinant nucleic acid encoding a light chain gene product that recognizes an epitope may be used to produce such an antibody fragment by recombinant means well known in the art.

In alternative embodiments of the instant invention, aromatic amino acids of protein binding elements may be replaced with other molecules. See U.S. Ser. Nos. 12/432,720 and 13/493,857 and U.S. Pat. No. 8,227,202.

In some embodiments, the activation state-specific binding element is a peptide comprising a recognition structure that binds to a target structure on an activatable protein. A variety of recognition structures are well known in the art and can be made using methods known in the art, including by phage display libraries (see e.g., Gururaja et al. Chem. Biol. (2000) 7:515-27; Houimel et al., Eur. J. Immunol. (2001) 31:3535-45; Cochran et al. J. Am. Chem. Soc. (2001) 123:625-32; Houimel et al. Int. J. Cancer (2001) 92:748-55, each incorporated herein by reference). Further, fluorophores can be attached to such antibodies for use in the methods of the present invention.

A variety of recognitions structures are known in the art (e.g., Cochran et al., J. Am. Chem. Soc. (2001) 123:625-32; Boer et al., Blood (2002) 100:467-73, each expressly incorporated herein by reference)) and can be produced using methods known in the art (see e.g., Boer et al., Blood (2002) 100:467-73; Gualillo et al., Mol. Cell Endocrinol. (2002) 190:83-9, each expressly incorporated herein by reference)), including for example combinatorial chemistry methods for producing recognition structures such as polymers with affinity for a target structure on an activatable protein (see e.g., Barn et al., J. Comb. Chem. (2001) 3:534-41; Ju et al., Biotechnol. (1999) 64:232-9, each expressly incorporated herein by reference). In another embodiment, the activation state-specific antibody is a protein that only binds to an isoform of a specific activatable protein that is phosphorylated and does not bind to the isoform of this activatable protein when it is not phosphorylated or nonphosphorylated. In another embodiment the activation state-specific antibody is a protein that only binds to an isoform of an activatable protein that is intracellular and not extracellular, or vice versa. In a some embodiment, the recognition structure is an anti-laminin single-chain antibody fragment (scFv) (see e.g., Sanz et al., Gene Therapy (2002) 9:1049-53; Tse et al., J. Mol. Biol. (2002) 317:85-94, each expressly incorporated herein by reference).

In some embodiments the binding element is a nucleic acid. The term “nucleic acid” include nucleic acid analogs, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments.

In some embodiment the binding element is a small organic compound. Binding elements can be synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or binding elements that can then be used in the present invention.

In some embodiments the binding element is a carbohydrate. As used herein the term carbohydrate is meant to include any compound with the general formula (CH20)n. Examples of carbohydrates are di-, tri- and oligosaccharides, as well polysaccharides such as glycogen, cellulose, and starches.

In some embodiments the binding element is a lipid. As used herein the term lipid is meant to include any water insoluble organic molecule that is soluble in nonpolar organic solvents. Examples of lipids are steroids, such as cholesterol, and phospholipids such as sphingomeylin.

In some embodiments, the binding elements are used to isolate the activatable elements prior to its detection, e.g. using mass spectrometry.

Examples of activatable elements, activation states and methods of determining the activation level of activatable elements are described in US publication number 20060073474 entitled “Methods and compositions for detecting the activation state of multiple proteins in single cells” and US publication number 20050112700 entitled “Methods and compositions for risk stratification” the content of which are incorporate here by reference.

### Labels

The methods and compositions of the instant invention provide detectable binding elements, e.g., binding elements comprising a label or tag. By label is meant a molecule that can be directly (i.e., a primary label) or indirectly (i.e., a secondary label) detected; for example a label can be visualized and/or measured or otherwise identified so that its presence or absence can be known. Binding elements and labels for binding elements are shown in See U.S. Ser. Nos. 12/432,720 and 13/493,857 and U.S. Pat. No. 8,227,202 and the other applications incorporated above.

A compound can be directly or indirectly conjugated to a label which provides a detectable signal, e.g. radioisotopes, fluorescers, enzymes, antibodies, particles such as magnetic particles, chemiluminescers, molecules that can be detected by mass spectrometry, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes. In some embodiments of the invention, these labels may be conjugated to the binding elements.

In some embodiments, one or more binding elements are uniquely labeled. Using the example of two activation state specific antibodies, by “uniquely labeled” is meant that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels fall into four classes: a) isotopic labels, which may be radioactive or heavy isotopes; b) magnetic, electrical, thermal labels; c) colored, optical labels including luminescent, phosphorous and fluorescent dyes or moieties; and d) binding partners. Labels can also include enzymes (horseradish peroxidase, etc.), magnetic particles, or mass tags. In some embodiments, the detection label is a primary label. A primary label is one that can be directly detected, such as a fluorophore.

Labels include optical labels such as fluorescent dyes or moieties. Fluorophores can be either “small molecule” fluors, or proteinaceous fluors (e.g. green fluorescent proteins and all variants thereof).

Labels also include mass labels such as mass tags, used in mass spectrometry.

In some embodiments, activation state-specific antibodies are labeled with quantum dots as disclosed by Chattopadhyay, P. K. et al. Quantum dot semiconductor nanocrystals for immunophenotyping by polychromatic flow cytometry. Nat. Med. 12, 972-977 (2006). Quantum dot labels are commercially available through Invitrogen, http://probes.invitrogen.com/products/qdot/.

Quantum dot labeled antibodies can be used alone or they can be employed in conjunction with organic fluorochrome-conjugated antibodies to increase the total number of labels available. As the number of labeled antibodies increase so does the ability for subtyping known cell populations. Additionally, activation state-specific antibodies can be labeled using chelated or caged lanthanides as disclosed by Erkki, J. et al. Lanthanide chelates as new fluorochrome labels for cytochemistry. J. Histochemistry Cytochemistry, 36:1449-1451, 1988, and U.S. Pat. No. 7,018,850, entitled Salicylamide-Lanthanide Complexes for Use as Luminescent Markers. Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy.

In some embodiments, the activatable elements are labeled with tags suitable for Inductively Coupled Plasma Mass Spectrometer (ICP-MS) as disclosed in Tanner et al. Spectrochimica Acta Part B: Atomic Spectroscopy, 2007 March; 62(3):188-195.

Alternatively, detection systems based on FRET, discussed in detail below, may be used. FRET finds use in the instant invention, for example, in detecting activation states that involve clustering or multimerization wherein the proximity of two FRET labels is altered due to activation. In some embodiments, at least two fluorescent labels are used which are members of a fluorescence resonance energy transfer (FRET) pair.

The methods and composition of the present invention may also make use of label enzymes. By label enzyme is meant an enzyme that may be reacted in the presence of a label enzyme substrate that produces a detectable product. Suitable label enzymes for use in the present invention include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By radioisotope is meant any radioactive molecule. Suitable radioisotopes for use in the invention include, but are not limited to 14C, 3H, 32P, 33P, 35S, 1251 and 1311. The use of radioisotopes as labels is well known in the art.

As mentioned, labels may be indirectly detected, that is, the tag is a partner of a binding pair. By “partner of a binding pair” is meant one of a first and a second moiety, wherein the first and the second moiety have a specific binding affinity for each other. Suitable binding pairs for use in the invention include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255: 192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto. As will be appreciated by those in the art, binding pair partners may be used in applications other than for labeling, as is described herein.

As will be appreciated, a partner of one binding pair may also be a partner of another binding pair. For example, an antigen (first moiety) may bind to a first antibody (second moiety) that may, in turn, be an antigen for a second antibody (third moiety). It will be further appreciated that such a circumstance allows indirect binding of a first moiety and a third moiety via an intermediary second moiety that is a binding pair partner to each.

As will be appreciated, a partner of a binding pair may comprise a label, as described above. It will further be appreciated that this allows for a tag to be indirectly labeled upon the binding of a binding partner comprising a label. Attaching a label to a tag that is a partner of a binding pair, as just described, is referred to herein as “indirect labeling”.

By “surface substrate binding molecule” or “attachment tag” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate [Field et al., Mol. Cell. Biol., 8:2159-2165 (1988)]; the c-myc tag and the 8F9, 3C7, 6E10, G4, B7 and 9E10 antibody substrates thereto [Evan et al., Molecular and Cellular Biology, 5:3610-3616 (1985)]; and the Herpes Simplex virus glycoprotein D (gD) tag and its antibody substrate [Paborsky et al., Protein Engineering, 3(6):547-553 (1990)]. In general, surface binding substrate molecules useful in the present invention include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

In some embodiments, the activatable elements are labeled by incorporating a label as describing herein within the activatable element. For example, an activatable element can be labeled in a cell by culturing the cell with amino acids comprising radioisotopes. The labeled activatable element can be measured using, for example, mass spectrometry.

### Alternative Activation State Indicators

An alternative activation state indicator useful with the instant invention is one that allows for the detection of activation by indicating the result of such activation. For example, phosphorylation of a substrate can be used to detect the activation of the kinase responsible for phosphorylating that substrate. Similarly, cleavage of a substrate can be used as an indicator of the activation of a protease responsible for such cleavage. Methods are well known in the art that allow coupling of such indications to detectable signals, such as the labels and tags described above in connection with binding elements. For example, cleavage of a substrate can result in the removal of a quenching moiety and thus allowing for a detectable signal being produced from a previously quenched label. In addition, binding elements can be used in the isolation of labeled activatable elements which can then be detected using techniques known in the art such as mass spectrometry.

### Detection

One or more activatable elements can be detected and/or quantified by any method that detects and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters. See U.S. Pat. No. 7,393,656 and Shulz et al., Current Protocols in Immunology, 2007, 78:8.17.1-20 which are incorporated by reference in their entireties.

In certain embodiments, the method of detection is flow cytometry or mass spectrometry. In certain embodiments, the method of detection is flow cytometry. In certain embodiments, the method of detection is mass spectrometry.

In practicing the methods of this invention, the detection of the status of the one or more activatable elements can be carried out by a person, such as a technician in the laboratory. Alternatively, the detection of the status of the one or more activatable elements can be carried out using automated systems. See U.S. Pat. Nos. 8,227,202 and 8,206,939 for some basic procedures and U.S. Ser. No. 12/606,869 for automation systems and procedures.

In some embodiments, the present invention provides methods for determining the activation level on an activatable element for a single cell. The methods may comprise analyzing cells by flow cytometry on the basis of the activation level at least one activatable element. Binding elements (e.g. activation state-specific antibodies) are used to analyze cells on the basis of activatable element activation level, and can be detected as described below. Binding elements can also be used to isolate activatable elements which can then be analyzed by methods known in the art. Alternatively, non-binding elements systems as described above can be used in any system described herein.

When using fluorescent labeled components in the methods and compositions of the present invention, different types of fluorescent monitoring systems, e.g., Cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells that may used as a modulator or as a population of reference cells. In some embodiments, the modulator or reference cells are first contacted with fluorescent-labeled binding elements (e.g. antibodies) directed against specific elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™. Training Manual, with particular reference to sections 3-11 to 3-28 and 10-1 to 10-17, which is hereby incorporated by reference in its entirety.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic filed while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods comprise providing a population of cells and analyze the population of cells by flow cytometry. Preferably, cells are analyzed on the basis of the activation level of at least one activatable element. In some embodiments, cells are analyzed on the basis of the activation level of at least two activatable elements.

In some embodiments, a multiplicity of activatable element activation-state antibodies is used to simultaneously determine the activation level of a multiplicity of elements.

In some embodiment, cell analysis by flow cytometry on the basis of the activation level of at least one activatable element is combined with a determination of other flow cytometry readable outputs, such as the presence of surface markers, granularity and cell. Similar determinations may be made by mass spectrometry, in which the elements are identified by mass tags rather than the fluorescent tags typical of flow cytometery. Any other suitable method known in the art may also be used, e.g., confocal microscopy.

As will be appreciated, these methods provide for the identification of distinct signaling cascades for both artificial and stimulatory conditions in cell populations, such a peripheral blood mononuclear cells, or naive and memory lymphocytes.

When necessary, cells are dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells may be fixed, e.g. with 3% paraformaldehyde, and are usually permeabilized, e.g. with ice cold methanol; HEPES-buffered PBS containing 0.1% saponin, 3% BSA; covering for 2 min in acetone at −200C; and the like as known in the art and according to the methods described herein.

In some embodiments, one or more cells are contained in a well of a 96 well plate or other commercially available multiwell plate. In an alternate embodiment, the reaction mixture or cells are in a cytometric measurement device. Other multiwell plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level of an activatable element, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element binds to the activatable element. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al. Spectrochimica Acta Part B: Atomic Spectroscopy, 2007 March; 62(3):188-195.). See also Bodenmiller et al, Nature Biotechnology, published online Aug. 19, 2012, doi:10.1038/nbt.2317.

As will be appreciated by one of skill in the art, the instant methods and compositions find use in a variety of other assay formats in addition to flow cytometry analysis. For example, a chip analogous to a DNA chip can be used in the methods of the present invention. Arrayers and methods for spotting nucleic acids on a chip in a prefigured array are known. In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell. See U.S. Pat. No. 5,744,934. In some embodiments, a microfluidic image cytometry is used (Sun et al. Cancer Res; 70(15) Aug. 1, 2010).

In some embodiments confocal microscopy can be used to detect activation profiles for individual cells. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, accordingly in this embodiment the cells can be labeled with one or more binding elements. In some embodiments the binding elements used in connection with confocal microscopy are antibodies conjugated to fluorescent labels, however other binding elements, such as other proteins or nucleic acids are also possible.

In some embodiments, the methods and compositions of the instant invention can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described below.

In some embodiments, the detecting is by high pressure liquid chromatography (HPLC), for example, reverse phase HPLC.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiments, the methods of the invention include the use of liquid handling components. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems. See U.S. Ser. No. 12/606,869 which is incorporated by reference in its entirety.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader. See U.S. Ser. No. 12/606,869.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory. See U.S. Ser. No. 12/606,869 which is incorporated by reference in its entirety.

These robotic fluid handling systems can utilize any number of different reagents, including buffers, reagents, samples, washes, assay components such as label probes, etc.

Any of the steps above can be performed by a computer program product that comprises a computer executable logic that is recorded on a computer readable medium. For example, the computer program can execute some or all of the following functions: (i) exposing different population of cells to one or more modulators, (ii) exposing different population of cells to one or more binding elements, (iii) detecting the activation levels of one or more activatable elements, and (iv) making a determination regarding the individual from whom the cells were collected, e.g., diagnosis, prognosis, categorization of disease, based on the activation level of one or more activatable elements in the different populations.

The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

The program can provide a method of determining the status of an individual by accessing data that reflects the activation level of one or more activatable elements in the reference population of cells.

### Analysis

Advances in flow cytometry have enabled the individual cell enumeration of up to thirteen simultaneous parameters (De Rosa et al., 2001) and are moving towards the study of genomic and proteomic data subsets (Krutzik and Nolan, 2003; Perez and Nolan, 2002). Likewise, advances in other techniques (e.g. microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. See Krutzik et al, Nature Chemical Biology February 2008. Methods for the analysis of multiple parameters are well known in the art. See U.S. Ser. Nos. 11/338,957, 12/910,769, 12/293,081, 12/538,643, 12/501,274 12/606,869 and PCT/2011/48332 for more information on analysis. See U.S. Ser. No. 12/501,295 for gating analysis.

In preparing a classifier for an end result, like a disease prediction, categorization, or prediction of drug response, the raw data from the detector, such as fluorescent intensity from a flow cytometer, is subject to processing using metrics outlined below. For simplicity, data is described in terms of fluorescent intensity but it will be understood that any data related to the activation level of an activatable protein may be analyzed by these methods. After treatment with the metrics, the data is fed to a model, such as machine learning, data mining, classification, or regression to provide a model for an outcome. There is also a selection of models to produce an outcome, which can be a prediction, prognosis, categorization, and the like.

The data can also be processed by using characteristics of cell health and cell maturity. Information on how to use cell health to analyze cells is shown in U.S. Ser. No. 61/436,534 and PCT/US2011/01565 which are incorporated by reference in their entireties. Restricting the analysis to cells that are not in active apoptosis can provide a more useful answer in the present assay. For example, in one embodiment, a method is provided to analyze cells comprising obtaining cells, determining if the cell is undergoing apoptosis and then excluding cells from a final analysis that are undergoing apoptosis. One way to determine if a cell is undergoing apoptosis is by measuring the intracellular level of one or more activatable elements related to cell health such as cleaved PARP, MCL-1, or other compounds whose activation state or activation level correlate to a level of apoptosis within single cells.

Indicators for cell health can include molecules and activatable elements within molecules associated with apoptosis, necrosis, and/or autophagy, including but not limited to caspases, caspase cleavage products such as dye substrates, cleaved PARP, cleaved cytokeratin 18, cleaved caspase, cleaved caspase 3, cytochrome C, apoptosis inducing factor (AIF), Inhibitor of Apoptosis (IAP) family members, as well as other molecules such as Bcl-2 family members including anti-apoptotic proteins (MCL-1, BCL-2, BCL-XL), BH3-only apoptotic sensitizers (PUMA, NOXA, Bim, Bad), and pro-apoptotic proteins (Bad, Bax) (see below), p53, c-myc proto-oncogene, APO-1/Fas/CD95, growth stimulating genes, or tumor suppressor genes, mitochondrial membrane dyes, Annexin-V, 7-AAD, Amine Aqua, trypan blue, propidium iodide or other viability dyes. In certain embodiments, cells are stained with Amine Aqua to distinguish viable from nonviable cells, and further stained with an indicator of apopotosis, e.g., an antibody to cPARP, to distinguish apoptosing from non-apoptosing cells.

Another general method for analyzing cells takes into account the maturity level of the cells. In one embodiment, cells that are immature (blasts) are included in the analysis and mature cells are not included. In another embodiment, the analysis can include all the patient's cells if they go above a certain threshold for the entire sample, for example, a call will be made on the basis of the entire sample. For example, samples having greater than 50, 60, 65, 70, 75, 80, 85, 90, or 95% immature cells can be classified as immature as a whole. In another embodiment, only those specific cells which are classified as immature are included in the analysis, irrespective of the total number of immature cells, for example, only those cells that are classified as immature will be part of the analysis for each sample. Or, a combination of the two methods could be employed, such as the counting of individual immature cells for samples that exceed a threshold related to cell maturity.

Cells may be classified as mature or immature manually or automatically. Methods for determining maturity are shown in Stelzer and Goodpasture, Immunophenotyping, 2000 Wiley-Liss Inc. which is incorporated by reference in its entirety. See also JOHN M. BENNETT, M. D., et al., Ann Intern Med. 1 Oct. 1985; 103(4):620-625.

In one embodiment, maturity may be determined by surface marker expression which can be applied to individual cells or at the sample level. The FAB system may also be used and applied to samples as a whole. For example, in one embodiment, samples as a whole are classified in the FAB system as M4, M5, or M7 are mature. In one embodiment, the cells may be analyzed by a variety of methods and markers, such as side scatter (SSC), CD11 b, CD117, CD45 and CD34. Generally, higher side scatter, and populations of CD45 or CD11b cells will indicate mature cells and generally lower populations of CD34 and CD117 will indicate mature cells. Immature populations are classified in the FAB system as M0, M1, M2 and M6. Generally, lower side scatter and populations of CD45 or CD11b cells will indicate immature cells and generally higher populations of CD34 and CD117 will indicate immature cells. Also, peripheral blood (PB) should have more mature cells than bone marrow (BM) samples. In some embodiments, analysis of the numbers or percentages of cells that can be classified as immature or mature will be necessary.

In one embodiment, cells are classified as mature or immature and then the immature cells are analyzed using a classifier. In another embodiment, the sample is classified as mature or immature and then the immature samples are analyzed using a classifier.

The metrics that are employed can relate to absolute cell counts, fluorescent intensity, frequencies of cellular populations (univariate and bivariate), relative fluorescence readouts (such as signal above background, etc.), and measurements describing relative shifts in cellular populations. In one embodiment, raw intensity data is corrected for variances in the instrument. Then the biological effect can be measured, such as measuring how much signaling is going on using the basal, fold, total and delta metrics. Also, a user can look at the number of cells that show signaling using the Mann Whitney model below.

In some embodiments where flow cytometry is used, flow cytometry experiments are performed and the results are expressed as fold changes using graphical tools and analyses, including, but not limited to a heat map or a histogram to facilitate evaluation. One common way of comparing changes in a set of flow cytometry samples is to overlay histograms of one parameter on the same plot. Flow cytometry experiments ideally include a reference sample against which experimental samples are compared. Reference samples can include normal and/or cells associated with a condition (e.g. tumor cells). See also U.S. Ser. No. 12/501,295 for visualization tools.

For example, the “basal” metric is calculated by measuring the autofluorescence of a cell that has not been stimulated with a modulator or stained with a labeled antibody. The “total phospho” metric is calculated by measuring the autofluorescence of a cell that has been stimulated with a modulator and stained with a labeled antibody. The “fold change” metric is the measurement of the total phospho metric divided by the basal metric. The quadrant frequency metric is the frequency of cells in each quadrant of the contour plot

A user may also analyze multimodal distributions to separate cell populations. Metrics can be used for analyzing bimodal and spread distribution. In some cases, a Mann-Whitney U Metric is used.

In some embodiments, metrics that calculate the percent of positive above unstained and metrics that calculate MFI of positive over untreated stained can be used.

A user can create other metrics for measuring the negative signal. For example, a user may analyze a “gated unstained” or ungated unstained autofluorescence population as the negative signal for calculations such as “basal” and “total”. This is a population that has been stained with surface markers such as CD33 and CD45 to gate the desired population, but is unstained for the fluorescent parameters to be quantitatively evaluated for node determination. However, every antibody has some degree of nonspecific association or “stickyness” which is not taken into account by just comparing fluorescent antibody binding to the autofluorescence. To obtain a more accurate “negative signal”, the user may stain cells with isotype-matched control antibodies. In addition to the normal fluorescent antibodies, in one embodiment, (phospho) or non phosphopeptides which the antibodies should recognize will take away the antibody's epitope specific signal by blocking its antigen binding site allowing this “bound” antibody to be used for evaluation of non-specific binding. In another embodiment, a user may block with unlabeled antibodies. This method uses the same antibody clones of interest, but uses a version that lacks the conjugated fluorophore. The goal is to use an excess of unlabeled antibody with the labeled version. In another embodiment, a user may block other high protein concentration solutions including, but not limited to fetal bovine serum, and normal serum of the species in which the antibodies were made, i.e. using normal mouse serum in a stain with mouse antibodies. (It is preferred to work with primary conjugated antibodies and not with stains requiring secondary antibodies because the secondary antibody will recognize the blocking serum). In another embodiment, a user may treat fixed cells with phosphatases to enzymatically remove phosphates, then stain.

In alternative embodiments, there are other ways of analyzing data, such as third color analysis (3D plots), which can be similar to Cytobank 2D, plus third D in color.

There are different ways to compare the distribution of X versus the distribution of Y. Examples are described below, such as Mann Whitney, U_{U}, fold change, and percent positive. There are also different biological processes to measure using the above metrics, such as modulated to unmodulated states, basal to autofluorescence, different cell types such as leukemic cell to lymphocytes, and mature as compared to immature cells.

Software may be used to examine the correlations among phosphorylation or expression levels of pairs of proteins in response to stimulus or modulation. The software examines all pairs of proteins for which phosphorylation and/or expression was measured in an experiment. The Total phosho metric (sometimes called “FoldAF”) is used to represent the phosphorylation or expression data for each protein; this data is used either on linear scale or log 2 scale.

For each protein pair under each experimental condition (unstimulated, stimulated, or treated with drug/modulator), the Pearson correlation coefficient and linear regression line fit are computed. The Pearson correlation coefficients for samples representing, e.g., responding and non-responding patients are calculated separately for each group and compared to the unperturbed (unstimulated) data. The following additional metrics are derived:
1. Delta CRNR unstim: the difference between Pearson correlation coefficients for each protein pair for the responding patients and for the non-responding patients in the basal or unstimulated state.
2. Delta CRNR stim: the difference between Pearson correlation coefficients for each protein pair for the responding patients and for the non-responding patients in the stimulated or treated state.
3. DeltaDelta CRNR: the difference between Delta CRNRstim and Delta CRNRunstim.

The correlation coefficients, line fit parameters (R, p-value, and slope), and the three derived parameters described above are computed for each protein-protein pair. Protein-protein pairs are identified for closer analysis by the following criteria:
1. Large shifts in correlations within patient classes as denoted by large positive or negative values (top and bottom quartile or 10^{th} and 90^{th} percentile) of the DeltaDelta CRNR parameter.
2. Large positive or negative (top and bottom quartile or 10^{th} and 90^{th} percentile) Pearson correlation for at least one patient group in either unstimulated or stimulated/treated condition.
3. Significant line fit (p-value <=0.05 for linear regression) for at least one patient group in either unstimulated or stimulated/treated condition.

All pair data is plotted as a scatter plot with axes representing phosphorylation or expression level of a protein. Data for each sample (or patient) is plotted with color indicating whether the sample represents a responder (generally blue) or non-responder (generally red). Further line fits for responders, non-responders and all data are also represented on this graph, with significant line fits (p-value <=0.05 in linear regression) represented by solid lines and other fits represented by dashed line, enabling rapid visual identification of significant fits. Each graph is annotated with the Pearson correlation coefficient and linear regression parameters for the individual classes and for the data as a whole. The resulting plots are saved in PNG format to a single directory for browsing using Picassa. Other visualization software can also be used.

In some embodiments a Mann Whitney statistical model is used for describing relative shifts in cellular populations. A Mann Whitney U test or Mann Whitney Wilcoxon (MWW) test is a non parametric statistical hypothesis test for assessing whether two independent samples of observations have equally large values. See Wikipedia at /http(colon)(slashslash)en.wikipedia.org(slash)wiki/Mann%E2%80%93Whitney_U/. The U metric may be more reproducible in some situations than Fold Change in some applications.

One example metric is Uᵤ. The Uᵤ is a measure of the proportion of cells that have an increase (or decrease) in protein levels upon modulation from the basal state. It is computed by dividing the scaled Mann-Whitney U statistic (/http(colonslashslash)en.wikipedia.org(slash)wiki/Mann % E2%80%93Whitney_U/) by the number of cells in the basal and the modulated populations. The cells in the two populations are ranked by the intensity values, only these ranks are then used to compute the statistic. As a result the metric is less sensitive to the absolute intensity values and depends only on relative shift between the two populations. The metric is bound between 0.0 and 1.0. A value of 0.5 would imply no shift in protein levels from the basal state, a value greater than 0.5 would imply an induction of signaling (i.e. increase in protein levels) and value less than 0.5 would imply an inhibition of signaling (i.e. decrease in protein levels).

$${U}_{u} = \frac{{R}_{m} - {n}_{m} ( {n}_{m} + 1 ) \text{/} 2}{{n}_{m} {n}_{u}}$$

Modulated (m) and unmodulated (u) populations are being compared
Rₘ=Sum of the ranks modulated population
nₘ=number of cells in the modulated population
nᵤ=number of cells in the unmodulated population

Uᵢ is another value that is the same as Uᵤ except that the isotype control is used as the reference instead of the unmodulated well.

TABLE 2
Examples of metrics

| Metric Class | Metric | Formal mathematics | Common usage |
|---|---|---|---|
| Absolute cell counts | Percent Recovery | $\frac{\# cells observed in a sample}{\# cells reported in sample vial}$ | Summary statistic describing the fraction of the cells that are observed |
| | | | after thawing and ficoll |
| | | | processing of |
| | | | cryopreserved cells |
| | Percent Viability | $\frac{\# cells Aqua negative}{total \# cells}$ | Summary statistic describing the fraction of the living cells that are |
| | | | observed from a given vial |
| | | | of samples. |
| | Percent Healthy | $\frac{\# cells Aqua negative and cPARP negative}{total \# cells}$ | Summary statistic describing the fraction of the living non-Apoptotic |
| | | | cells that are observed |
| | | | from a given vial of |
| | | | samples. |
| Fluorescence | MFI (Median | | A summary statistic |
| Intensity | Fluorescence | | (median) of the non- |
| Metrics | Intensity) | | calibrated intensity of |
| | | | particular fluorescence |
| | | | readouts |
| | ERF | | Used to describe the |
| | (Equivalent | | fluorescence intensity |
| | Reference | | readout as calibrated for |
| | Fluorescence) | | the specific instrument on |
| | | | the specific date of usage. |
| | | | Can be applied at the |
| | | | single cell level or to bulk |
| | | | properties of cellular |
| | | | populations. See U.S. Pat. |
| | | | No. 8,187,885. |
| Frequencies of cellular populations- | Percent of Cells | $\frac{Number cells of interest}{Number cells Total population}$ | Describes the fraction of cells of a given type relative to the population. |
| univariate | | | Can be defined as a one- |
| | | | dimensional or 2- |
| | | | dimensional region or gate |
| | Percentage Positive | $\frac{\# cells > Cutoff}{Number cells Total population}$ | Describes the portion of cells above a given threshold (I.e. a control |
| | | | antibody) of single assay |
| | | | readout |
| Frequencies of cellular populations- | Quadrant gate “Quad” | $\frac{Number cells of interest in each quadrant}{Number cells Total population}$ | Quantitative measure of the percentage of cells in each one of four regions of |
| bivariate | | | interest. |
| Fold | Basal | ${log}_{2} \frac{{ERF}_{unmodulated}}{{ERF}_{autofluorescence}}$ | Describes the magnitude of the activation levels of signaling in the resting, |
| | | | unmodulated state. This |
| | | | metric is corrected to |
| | | | accommodate the |
| | | | background |
| | | | autofluorescence and |
| | | | instrument noise. |
| | Modulated | ${log}_{2} \frac{{ERF}_{modulated}}{{ERF}_{unmodulated}}$ | Describes the magnitude of the inducibility or responsiveness of a protein |
| | | | or a signaling pathway |
| | | | activation response to |
| | | | modulation. This metric is |
| | | | always calculated relative |
| | | | to the unmodulated (basal) |
| | | | level of activation. |
| | | | Autofluorescence and |
| | | | instrument noise do not |
| | | | appear in the equation |
| | | | since they appear in both |
| | | | the numerator and |
| | | | denominator (CHECK) |
| | Total | ${log}_{2} \frac{{ERF}_{modulated}}{{ERF}_{autofluorescence}}$ | Used to assess the magnitude of total activated protein. This |
| | | | metric incorporates both |
| | | | basal and induced pathway |
| | | | activation. |
| | Relative Protein Expression | ${log}_{2} \frac{{ERF}_{Expression Marker}}{{ERF}_{isotype control}}$ | Used to measure the amount of surface expression of a particular |
| | “Rel | | protein. In this case, the |
| | Expression” | | metric is always calculated |
| | | | relative to the background |
| | | | level of an isotype control |
| | | | and instrument noise. |
| Mann- Whitney U Metrics | Uₐ | $\frac{{R}_{u} - {n}_{u} ( {n}_{u} + 1 ) / 2}{{n}_{u} {n}_{a}}$ | This is a rank-based metric. It is used to describe the shift in a |
| | | Unmodulated (u) and | population of cells in an |
| | | autofluorescence (a) | unmodulated state relative |
| | | populations are being | to the population seen in |
| | | compared. | the autofluorescence |
| | | Rᵤ = Sum of the ranks | (background). All single |
| | | unmodulated population | cell events are used in the |
| | | nᵤ = number of cells in the | calculation. |
| | | unmodulated population | It is formally a scaled |
| | | nₐ = number of cells in the | Mann-Whitney U metric |
| | | autofluorescence population | (AUC). |
| | Uᵤ | $\frac{{R}_{m} - {n}_{m} ( {n}_{m} + 1 ) / 2}{{n}_{m} {n}_{u}}$ | This is a rank-based metric. It is used to describe the shift in a |
| | | Modulated (m) and | population of cells in a |
| | | unmodulated (u) populations | modulated state relative to |
| | | are being compared. | the population seen in the |
| | | Rₘ = Sum of the ranks | unmodulated (basal) state. |
| | | unmodulated population | All single cell events are |
| | | nₘ = number of cells in the | used in the calculation. |
| | | modulated population | It is formally a scaled |
| | | nᵤ = number of cells in the | Mann-Whitney U metric |
| | | unmodulated population | (AUC). |
| | Percent | | Used to describe the ability |
| | Inhibition | | of a compound or other |
| | | | agent to modify the |
| | | | activity levels (assuming |
| | | | decreased activation) of a |
| | | | given measure (e.g. MFI, |
| | | | ERF, Uᵤ, etc.) |

Each protein pair can be further annotated by whether the proteins comprising the pair are connected in a “canonical” pathway. In the current implementation canonical pathways are defined as the pathways curated by the NCI and Nature Publishing Group. This distinction is important; however, it is likely not an exclusive way to delineate which protein pairs to examine. High correlation among proteins in a canonical pathway in a sample may indicate the pathway in that sample is “intact” or consistent with the known literature. One embodiment of the present invention identifies protein pairs that are not part of a canonical pathway with high correlation in a sample as these may indicate the non-normal or pathological signaling. This method is used to identify stimulator/modulator-stain-stain combinations that distinguish classes of patients.

In some embodiments, nodes and/or nodes/metric combinations can be analyzed and compared across sample for their ability to distinguish among different groups (e.g., CR vs. NR patients) using classification algorithms. Any suitable classification algorithm known in the art can be used. Examples of classification algorithms that can be used include, but are not limited to, multivariate classification algorithms such as decision tree techniques: bagging, boosting, random forest, additive techniques: regression, lasso, bblrs, stepwise regression, nearest neighbors or other methods such as support vector machines.

In some embodiments, nodes and/or nodes/metric combinations can be analyzed and compared across sample for their ability to distinguish among different groups (e.g., CR vs. NR patients) using random forest algorithm. Random forest (or random forests) is an ensemble classifier that consists of many decision trees and outputs the class that is the mode of the class's output by individual trees. The algorithm for inducing a random forest was developed by Leo Breiman (Breiman, Leo (2001). “Random Forests”. Machine Learning 45 (1): 5-32. doi:10.1023/A:1010933404324) and Adele Cutler. The term came from random decision forests that was first proposed by Tin Kam Ho of Bell Labs in 1995. The method combines Breiman's “bagging” idea and the random selection of features, introduced independently by Ho (Ho, Tin (1995). “Random Decision Forest”. 3rd Int'l Conf. on Document Analysis and Recognition. pp. 278-282; Ho, Tina (1998). “The Random Subspace Method for Constructing Decision Forests”. IEEE Transactions on Pattern Analysis and Machine Intelligence 20 (8): 832-844. doi:10.1109/34.709601) and Amit and Geman (Amit, Y.; Geman, D. (1997). “Shape quantization and recognition with randomized trees”. Neural Computation 9 (7): 1545-1588. doi:10.1162/neco.1997.9.7.1545) in order to construct a collection of decision trees with controlled variation.

In some embodiments, nodes and/or nodes/metric combinations can be analyzed and compared across sample for their ability to distinguish among different groups (e.g., CR vs. NR patients) using lasso algorithm. The method of least squares is a standard approach to the approximate solution of overdetermined systems, i.e. sets of equations in which there are more equations than unknowns. “Least squares” means that the overall solution minimizes the sum of the squares of the errors made in solving every single equation. The best fit in the least-squares sense minimizes the sum of squared residuals, a residual being the difference between an observed value and the fitted value provided by a model.

In some embodiments, nodes and/or nodes/metric combinations can be analyzed and compared across sample for their ability to distinguish among different groups (e.g., CR vs. NR patients) using BBLRS model building methodology.

a. Description of the BBLRS Model Building Methodology

Production of Bootstrap Samples:

A large number of bootstrap samples are first generated with stratification by outcome status to insure that all bootstrap samples have a representative proportion of outcomes of each type. This is particularly important when the number of observations is small and the proportion of outcomes of each type is unbalanced. Stratification under such a scenario is especially critical to the composition of the out of bag (OOB) samples, since only about one-third of observations from the original sample will be included in each OOB sample.

Best Subsets Selection of Main Effects:

Best subsets selection is used to identify the combination of predictors that yields the largest score statistic among models of a given size in each bootstrap sample. Models having from 1 to 2×N/10 are typically entertained at this stage, where N is the number of observations. This is much larger than the number of predictors generally recommended when building a generalized linear prediction model (Harrell, 2001) but subsequent model building rules are applied to reduce the likelihood of over-fitting. At the conclusion of this step, there will be a “best” main effects model of each size for each bootstrap sample, though the number of unique models of each size may be considerably fewer.

Determination of the Optimal Model Size (for Main Effects):

Each of the unique “best” models of each size, identified in the previous step, are fit to each of a subset of the bootstrap samples, where the number of bootstrap samples in the subset is under the control of the user (i.e. a tuning parameter) so that the processing time required at this step can be controlled. For each of the bootstrap samples in the subset, the median SBC of the “best” models of the same size is calculated and the model size yielding the lowest median SBC in that bootstrap sample is identified. The optimal model size is then determined as the size for which the median SBC is smallest most often over the subset of bootstrap samples.

Identification of the Top Models of the Best Size:

At this stage, all previously identified “best” models of the optimal size are fit to every bootstrap sample. A number of top models are then selected as those with the highest values of the margin statistic (a measure from the logistic model of the difference in the predicted probabilities of CR, between NR patients with the highest predicted probabilities and CR patients with the lowest predicted probabilities). In order to limit the processing time required in subsequent steps, the number of top models selected is under the control of the user.

Identification of Important Two-Wav Interactions:

For each of the top main effects models identified in the previous step, models are constructed on every bootstrap sample, with main effects forced into the model and with stepwise selection used to identify important two-way interactions among the set of all possible pair-wise combinations of the main effects. The nominal significance level for entry and removal of interaction terms is under the control of the user. Significance levels greater than 0.05 are often used for entry because of the low power many studies have to detect interactions and because safeguards against over-fitting are applied subsequently.

At this stage, collections of full models (main effects and possibly some two-way interactions among them) have been constructed (on the set of all bootstrap samples) for each unique set of main effects identified in the previous step. The top full models in each collection are then chosen as those constructed most frequently over all bootstrap samples, where winners are decided among tied models by the lowest mean SBC and then the highest mean AUROC. The number of full models in each collection that are advanced to the next step is under the control of the user.

Selection of the Effects in the Final Model:

Each full model advanced to this step is fit to every bootstrap sample and the median margin statistic for each model over the bootstrap samples is calculated. The model with the highest median margin statistic is selected as the final model. If there are ties, the model with the lowest mean SBC is selected.

Technically, the procedure described here results in the selection of the effects (main effects and possibly two-way interactions) to be included in the final model, but not specification of the model itself. The latter includes the effects and the specific regression coefficients associated with the intercept and each of the model effects.

Specification of the Final Model:

The effects in the final model are then fit to the complete dataset using Firth's method to apply shrinkage to the regression coefficient estimates. The model effects and their estimated regression coefficients (plus the estimate of the intercept) comprise the final model.

Another method of the present invention relates to display of information using scatter plots. Scatter plots are known in the art and are used to visually convey data for visual analysis of correlations. See U.S. Pat. No. 6,520,108. The scatter plots illustrating protein pair correlations can be annotated to convey additional information, such as one, two, or more additional parameters of data visually on a scatter plot.

Previously, scatter plots used equal size plots to denote all events. However, using the methods described herein two additional parameters can be visualized as follows. First, the diameter of the circles representing the phosphorylation or expression levels of the pair of proteins may be scaled according to another parameter. For example they may be scaled according to expression level of one or more other proteins such as transporters (if more than one protein, scaling is additive, concentric rings may be used to show individual contributions to diameter).

Second, additional shapes may be used to indicate subclasses of patients. For example they could be used to denote patients who responded to a second drug regimen or where CRp status. Another example is to show how samples or patients are stratified by another parameter (such as a different stim-stain-stain combination). Many other shapes, sizes, colors, outlines, or other distinguishing glyphs may be used to convey visual information in the scatter plot.

In this example the size of the dots is relative to the measured expression and the box around a dot indicates a NRCR patient that is a patient that became CR (Responsive) after more aggressive treatment but was initially NR (Non-Responsive). Patients without the box indicate a NR patient that stayed NR.

In some embodiments, analyses are performed on healthy cells. The health of the cells can be determined by using cell markers that indicate cell health. Cells that are dead and/or undergoing apoptosis can be removed from the analysis. In some embodiments, cells are stained with apoptosis and/or cell death markers such as PARP or Aqua dyes. Cells undergoing apoptosis and/or cells that are dead can be gated out of the analysis. In some embodiments, the measurements of activatable elements are adjusted by measurements of sample quality for the individual sample, such as the percent of healthy cells present.

A regression equation can be used to adjust raw node readout scores for the percentage of healthy cells at 24 hours post-thaw. Means and standard deviations can be used to standardize the adjusted node readout scores.

Before applying the SCNP classifier, raw node-metric signal readouts (measurements) for samples can be adjusted for the percentage of healthy cells and then standardized. The adjustment for the percentage of healthy cells and the subsequent standardization of adjusted measurements is applied separately for each of the node-metrics in the SCNP classifier.

The following formula can be used to calculate the adjusted, normalized node-metric measurement (z) for each of the node-metrics of each sample.

z=((x−(b₀+bᵢₓ pcthealthy))−residual_mean)/residual_sd,

where x is the raw node-metric signal readout, b₀ and b₁ are the coefficients from the regression equation used to adjust for the percentage of healthy cells (pcthealthy), and residual_mean and residual_sd are the mean and standard deviation, respectively, for the adjusted signal readouts in the training set data. The values of b₀, b₁, residual_mean, and residual_sd for each node-metric are included in the embedded object below, with values of the latter two parameters stored in variables by the same name. The values of the b₀ and b₁ parameters are contained on separate records in the variable named “estimate”. The value for b₀ is contained on the record where the variable “parameter” is equal to “Intercept” and the value for b₁ is contained on the record where the variable “parameter” is equal to “percenthealthy24Hrs”. The value of pcthealthy will be obtained for each sample as part of the standard assay output. The SCNP classifier will be applied to the z values for the node-metrics to calculate the continuous SCNP classifier score and the binary induction response assignment (pNR or pCR) for each sample.

In some embodiments, the measurements of activatable elements are adjusted by measurements of sample quality for the individual cell populations or individual cells, based on markers of cell health in the cell populations or individual cells. Examples of analysis of healthy cells can be found in U.S. Application Ser. No. 61/374,613 filed Aug. 18, 2010, PCT/US2011/001565, and PCT/US2011/048332 the contents of which are incorporated herein by reference in its entirety for all purposes.

### Kits

In some embodiments the invention provides kits. Kits provided by the invention may comprise one or more of the state-specific binding elements described herein, such as phospho-specific antibodies. A kit may also include other reagents that are useful in the invention, such as modulators, fixatives, containers, plates, buffers, therapeutic agents, instructions, and the like.

In some embodiments, the kit comprises one or more of the phospho-specific antibodies specific for the proteins selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHPT, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLCγ1, PLCγ2, STAT1, STAT 3, STAT 4, STAT 5, STAT 6, FAK, p130CAS, PAKs, LIMK1/2, Hsp90, Hsp70, Hsp27, SMADs, Rel-A (p65-NFκB), CREB, Histone H2B, HATs, HDACs, PKR, Rb, Cyclin D, Cyclin E, Cyclin A, Cyclin B, P16, pl4Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(RelA), IKKα, PKA, PKCα, PKCβ, PKCθ, PKCδ, CAMK, Elk, AFT, Myc, Egr-1, NFAT, ATF-2, Mdm2, p53, DNA-PK, Chk1, Chk2, ATM, ATR, (3-catenin, CrkL, GSK3α, GSK3β, and FOXO. In some embodiments, the kit comprises one or more of the phospho-specific antibodies specific for the proteins selected from the group consisting of Erk, Syk, Zap70, Lck, Btk, BLNK, Cbl, PLCγ2, Akt, RelA, p38, S6. In some embodiments, the kit comprises one or more of the phospho-specific antibodies specific for the proteins selected from the group consisting of Akt1, Akt2, Akt3, SAPK/JNK1,2,3, p38s, Erk1/2, Syk, ZAP70, Btk, BLNK, Lck, PLCγ, PLCγ2, STAT1, STAT 3, STAT 4, STAT 5, STAT 6, CREB, Lyn, p-S6, Cbl, NF-κB, GSK3β, CARMA/Bcl10 and Tcl-1.

The state-specific binding element of the invention can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Such kits enable the detection of activatable elements by sensitive cellular assay methods, such as IHC and flow cytometry, which are suitable for the clinical detection, prognosis, and screening of cells and tissue from patients, such as leukemia patients, having a disease involving altered pathway signaling.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### EXAMPLES

### Example 1

### Functional Analysis of Interferon Responsiveness in PBMC from SLE Donors Identifies Subgroups with Higher and Lower Disease Activity

Interferons (IFN) reportedly are central to SLE pathogenesis and increased expression of IFN regulated genes (the ‘IFN signature’) is associated with active disease. Clinical utility of the IFN signature is unclear, and refinement to define further patient subgroups may improve disease management. Toll-like receptor (TLR) activation leads to IFNα induction. To increase understanding of the role of IFNs in SLE pathobiology, and connectivity between IFN and TLR signaling, functional profiling of immune signaling downstream of IFNα, IFNγ and TLR modulators in peripheral blood mononuclear cells (PBMC) of SLE donors was performed and compared with signaling in healthy donors (HD).

Methods:

Single Cell Network Profiling (SCNP) is a multiparametric flow cytometry based technology that enables simultaneous analysis of signaling networks in multiple immune cell subsets. PBMC from 60 SLE patients (meeting ACR criteria (2007), SELENA SLEDAI ≧6) and 59 HD were profiled by SCNP, interrogating IFN modulated JAK-STAT signaling and TLR modulated signaling relevant to SLE. CD4+/−CD45RA+/− T cells, CD20+ B cells, CD14+ monocytes and CD11b+ myeloid dendritic cells were profiled, (see Table 1). Donor demographics are given in Table 2.

TABLE 1
Modulators, readouts, and cell subsets analyzed

| Modulator | Intracellular Reads | Cell Subsets Analyzed |
|---|---|---|
| IFNα | p-STAT1, p-STAT3, | B cells, Monocytes, |
| | p-STAT5 | T cell subsets |
| IFNγ | p-STAT1, p-STAT3, | B cells, Monocytes, |
| | p-STAT5 | T cell subsets |
| Pam3CSK4 (TLR1/2) | p-ERK, p-p38, Ikb, | Monocytes |
| | p-c-Jun, p-CREB | |
| LPS (TLR4) | p-ERK, p-p38, Ikb, | Monocytes |
| | p-c-Jun, p-CREB | |
| R848 (TLR7/8) | p-ERK, p-p38, Ikb, | B cells, Monocytes, |
| | p-c-Jun, p-CREB | mDCs |
| CpG-C (TLR9) | p-AKT, p-ERK, p-S6, | B cells |
| | IkB, p-STAT3 | |

TABLE 2
Donor Demographics
DONOR DEMOGRAPHICS

| Character- | | | | | |
|---|---|---|---|---|---|
| istics and | | Disease | | Healthy | |
| Demographics | Values | (n = 60) | | (n = 59) | |
| Age | 18 to 19 years, n (%) | 2 | (3.3) | 3 | (5.1) |
| | 20 to 29 years, n (%) | 9 | (15) | 15 | (25.4) |
| | 30 to 39 years, n (%) | 11 | (18.3) | 11 | (18.6) |
| | 40 to 49 years, n (%) | 16 | (26.7) | 13 | (22.0) |
| | 50 to 59 years, n (%) | 13 | (21.7) | 15 | (25.4) |
| | 60+ years, n (%) | 9 | (15) | 2 | (3.4) |
| Race | Caucasian, n (%) | 37 | (61.7) | 35 | (59.3) |
| | African American, n (%) | 13 | (21.7) | 17 | (28.8) |
| | Asian, n (%) | 8 | (13.3) | 4 | (6.8) |
| | Others, n (%} | 2 | (3.3) | 3 | (5.1) |
| Gender | Female, n (%) | 55 | (91.7) | 57 | (94.9) |
| | Male, n (%) | 5 | (8.3) | 3 | (5.1) |
| Medication | Aspirin, n (%) | 8 | (13.3) | 5 | (8.5) |
| | Diabetes Medication | 2 | (3.3) | 2 | (3.4) |
| | Thyroid Replacement, | 9 | (15) | 3 | (5.1) |
| | n (%) | | | | |
| | Hormones, n (%) | 10 | (16.7) | 5 | (8.5) |
| | Statins, n (%) | 9 | (15) | 4 | (6.8) |

| | | | | |
|---|---|---|---|---|
| | Anti-malarial drugs, | 40 | (67) | NA |
| | n (%) | | | |
| | Belimumab, n (%) | 16 | (26.7) | NA |
| SLE | SELENA-SLEDAI | 6-16 | (8.5) | NA |
| Character- | Score Range | | | |
| istics | (Average) | | | |
| | Positive ANA, n (%) | 53 | (88.3) | NA |
| | Positive anti-SM, n (%) | 8 | (13.3) | NA |
| | Positive anti-dsDNA, | 32 | (53.3) | NA |
| | n (%) | | | |
| | Low Complements, | 18 | (30) | NA |
| | n (%) | | | |
| | Anemia, n (%) | 23 | (38.3) | NA |
| | Proteinuria, n (%) | 9 | (15) | NA |

Inclusion criteria for enrollment
18 to 65 years of age
Diagnosis of SLE by a minimum of 4 out of 11 ACR criteria, one of which must be an ANA with a titer of 1:180 or greater or the presence of anti-dsDNA or anti-Sm Abs
SLEDAI score ≧ 6
Stable SLE treatment for the 30 days preceding blood collection
One or more elevated autoantibody levels in the preceding year

### Results:

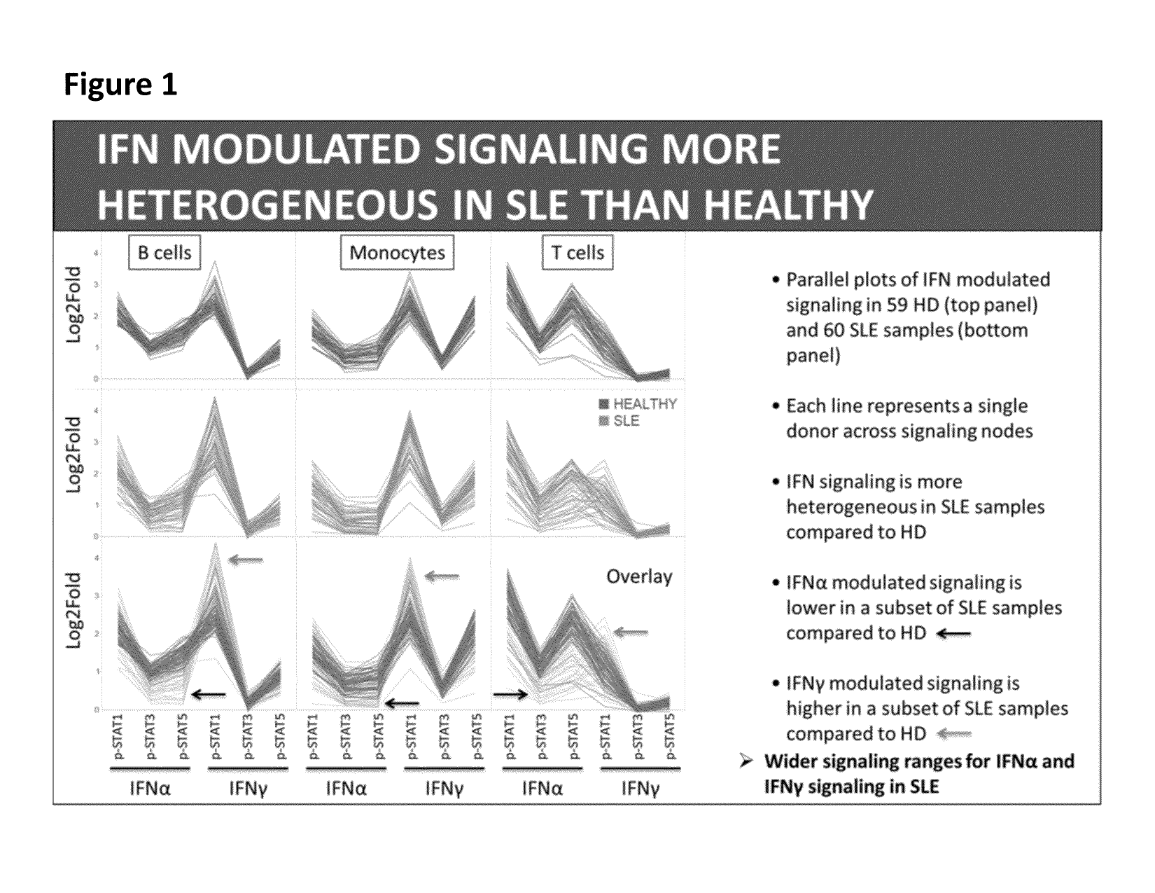

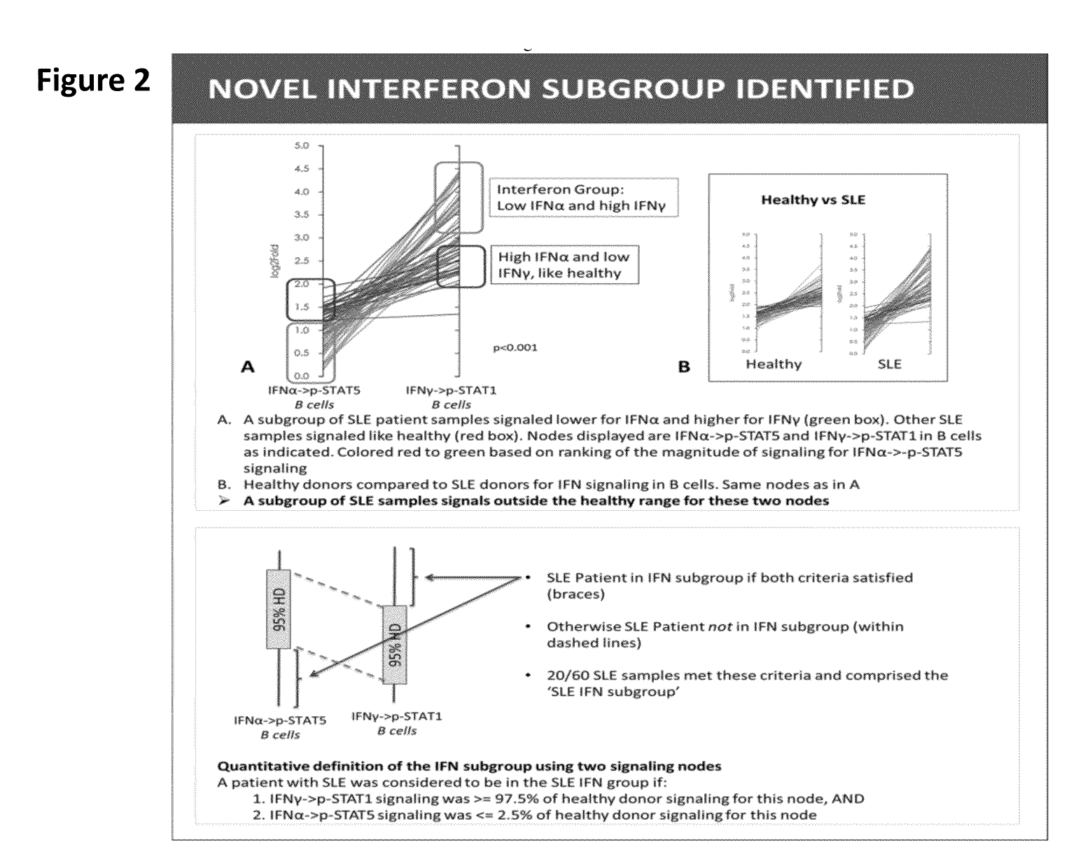

IFNα and IFNγ modulated p-STAT1, -3 and -5 signaling was more heterogeneous in SLE vs HD. See FIG. 1. An SLE subgroup demonstrated low IFNα/high IFNγ signaling in lymphocytes and monocytes. See FIG. 2. Based on low IFNα→p-STAT5/high IFNγ→p-STAT1 modulated signaling in B cells, the SLE-IFN subgroup was defined as outside the 95 percentile (z-score>+/−1.96) of HD, comprising 20 of 60 SLE samples. See FIG. 2.

The SLE-IFN subgroup was 9.4-fold more likely to be positive for anti-dsDNA antibodies (Fisher's exact test p-val<0.001), consistent with published data on the IFN signature and its link to disease activity, and supporting the clinical relevance of this observation. Significant associations with ANA Ab positivity (p=0.04), report of a new rash (p=0.03) and age (p=0.04) were also identified. No significant associations with other clinical or demographic parameters were identified.

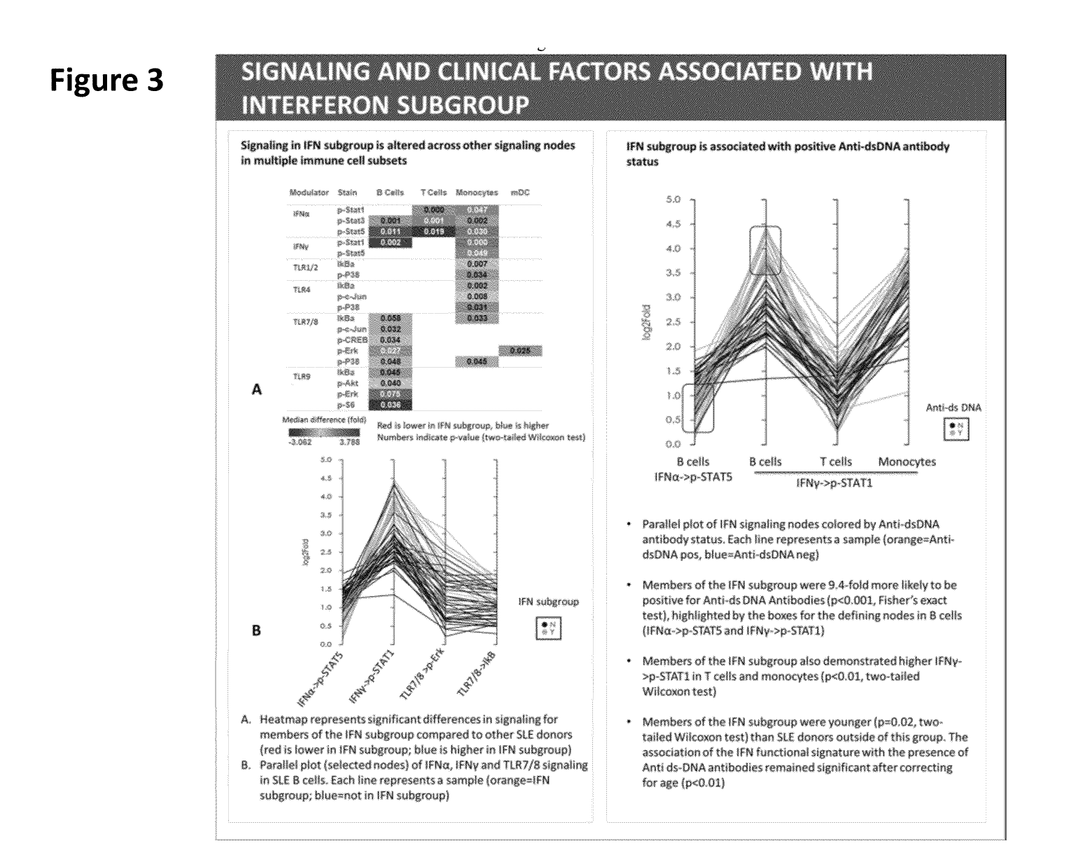

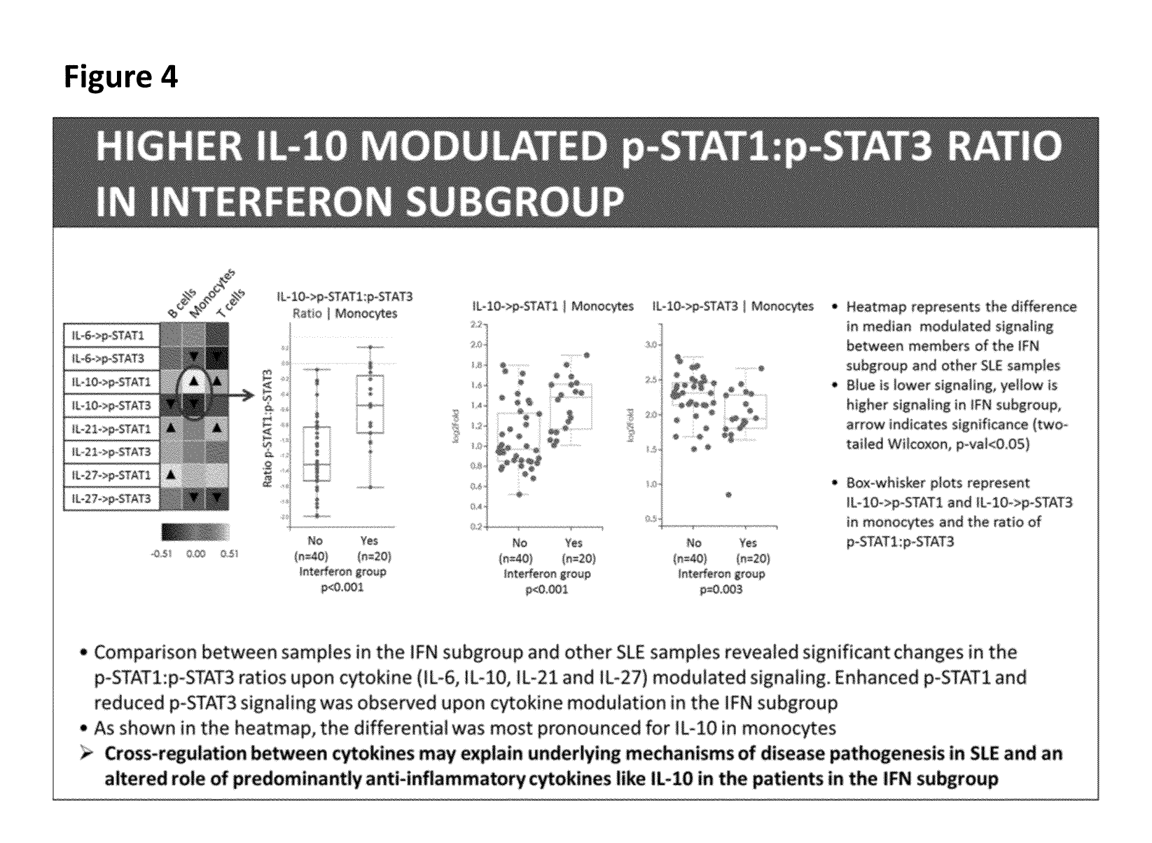

Strikingly, the members of the SLE-IFN subgroup displayed higher TLR7/8 modulated signaling in B cells (Wilcoxon test p=0.003-0.03, depending on the intracellular readout), and dendritic cells (p=0.03), but not in monocytes. Moreover, TLR9 signaling was lower in B cells (p=0.02), and TLR1/2 and TLR4 modulated signaling was lower in monocytes (p=0.003-0.01). See FIG. 3. In addition, comparison between samples in the IFN subgroup and other SLE samples revealed significant changes in the p-STAT1:p-STAT3 ratios upon cytokine (IL-6, IL-10, IL-21, and IL-27) modulated signaling. Enhanced p-STAT-1 and reduced p-STAT3 signaling was observed upon cytokine modulation in the IFN subgroup. See FIG. 4.

Conclusion:

These data identify potential connectivity in immune signaling across cell subsets and signaling pathways that underlie disease pathobiology and further define SLE donor subgroups. Refinement of the IFN signature in SLE through SCNP may facilitate the clinical applicability of the signature to better inform patient stratification for treatment options.

SCNP analysis of 60 SLE and 59 healthy donor PBMCs has identified an immune signaling signature that differentiates an SLE donor subgroup (n=20) from healthy donors through IFNα→p-STAT5 and IFNg→p-STAT1 in B cells, and this SLE IFN subgroup was associated positively with the presence of Anti-dsDNA antibodies. Additional signaling nodes across immune cell subsets associated with this signature, suggesting the possibility to define the mechanistic basis of this signaling profile and further define categories within the overall IFN subgroup. The cytokine modulated p-STAT1:3 ratio was higher in the SLE IFN subgroup, suggesting cross-regulation between cytokines and demonstrating the interaction if innate and adaptive immune responses. These data are supportive of the application of SCNP to interrogate the basis of SLE-associated signaling and may facilitate the clinical applicability of the signature to better inform patient stratification for treatment options, identify new points of intervention and potential combinatorial therapies in SLE patient subgroups.

### Example 2

### Functional Profiling of PBMC from SLE Patients Versus Healthy Controls Identifies Subgroups with Disease-Associated Dysfunctional Signaling

Systemic Lupus Erythematosus (SLE) is a complex multi-system rheumatic disease with widely differing clinical manifestations and outcomes. Treatment is often symptom directed or generally immunosuppressive, with no available biomarkers to inform therapeutic selection for a given patient or disease manifestation. Profiling the immune signaling pathways in PBMCs from patients with active SLE and healthy donors (HD) enables improved understanding of pathobiology and provides a basis for rational treatment decisions.

Methods:

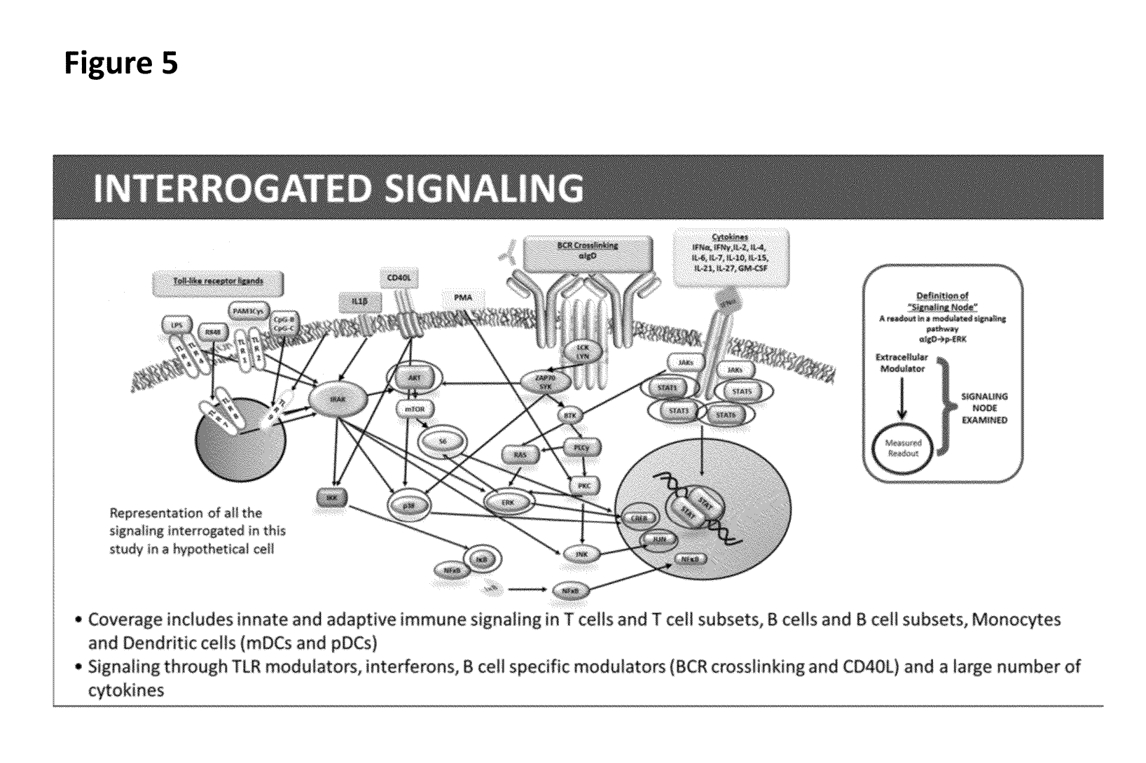

Single Cell Network Profiling (SCNP) is a multiparametric flow cytometry based technology that enables simultaneous quantitative analysis of signaling networks in multiple immune cell subsets. PBMC from 60 SLE patients meeting ACR (2007) criteria with SELENA-SLEDAI scores ≧6 were profiled by SCNP and compared to PBMC from 59 age, gender and race matched HD in the presence and absence of modulators of immune function (11 cytokines; 5 toll-like receptor (TLR) modulators and IL-1β; B cell-specific modulators CD40L and Anti-IgD, and PMA), across B (defined by IgD and CD27) and T (CD4/CD45RA) cell subsets, monocytes, and dendritic (HLA-DR, CD11b, CD123) cells, and evaluated through induced p-STATs, MAPK, PI3K and NFκB pathway readouts. FIG. 5 shows the signaling nodes interrogated in the study. Donor demographics were as shown in Table 2 of Example 1.

Results:

SLE vs HD:

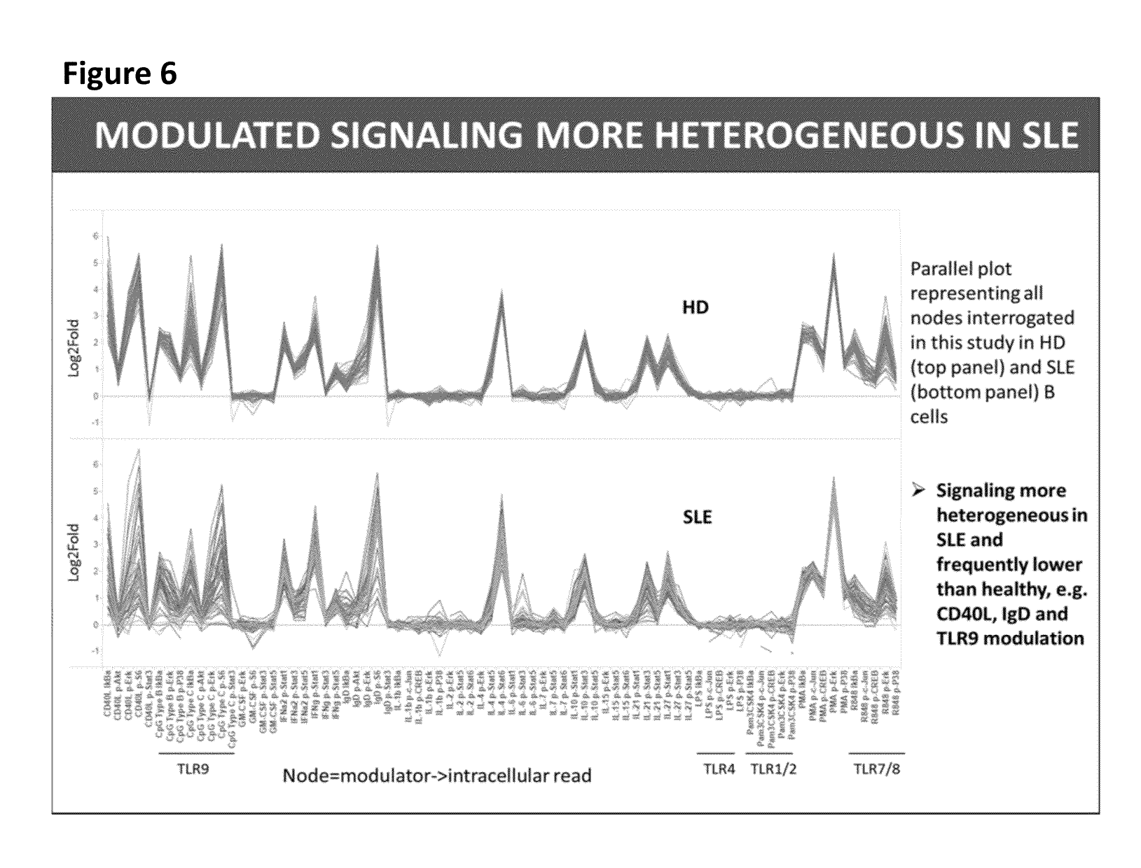

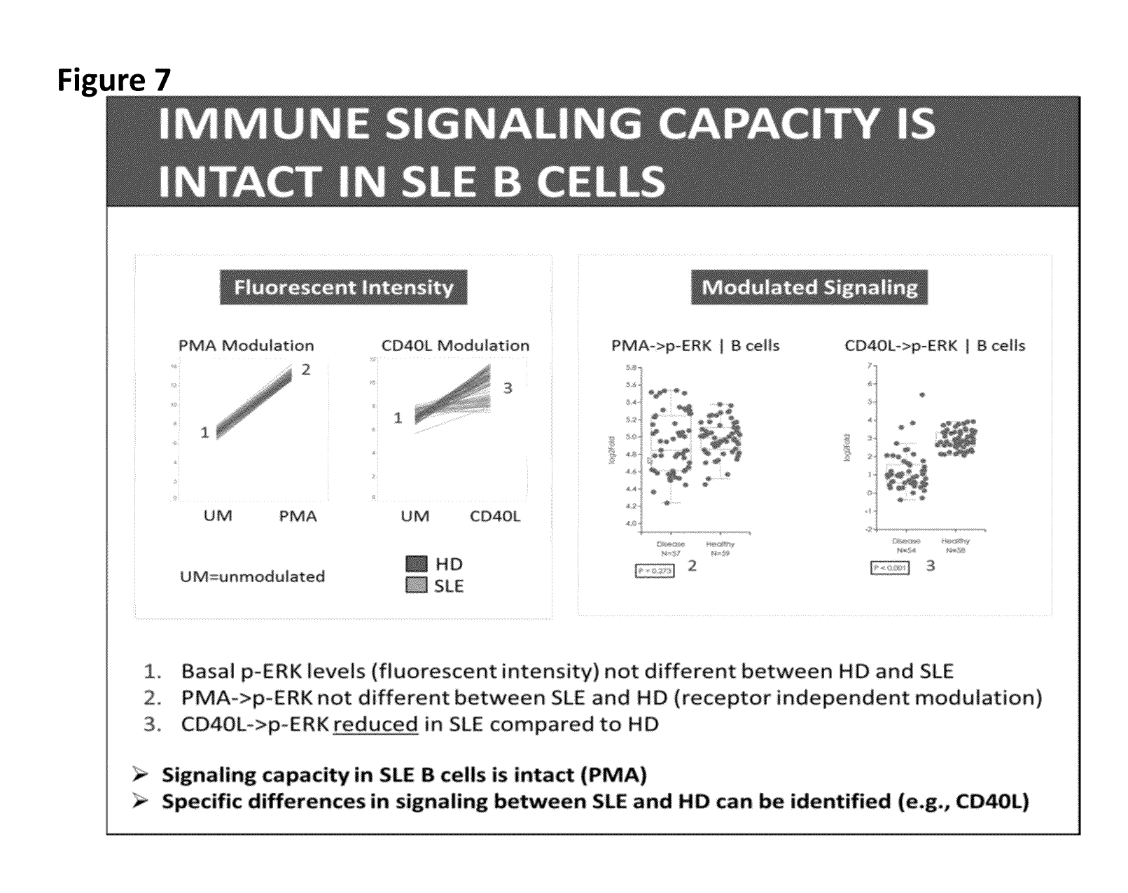

SLE PBMC overall had a broader signaling range than HD, with median modulated signaling in B and T cells lower in SLE. See FIG. 6. Exceptions include IFNγ→p-STAT1 in B cells and CD45RA+CD4+ T cells, IL-2→p-STAT5 in CD45RA+CD4+ T cells, IL-4→p-STAT6 in T cell subsets, and IL-10→p-STAT1, -3 in T cell subsets. Modulation of p-STAT1 by IFNγ, IL-10 and IL-27, and IL-6→p-STAT3 was increased in SLE monocytes. TLR→p-ERK, but not NFκB signaling was increased in monocytes. SLE mDCs showed elevated TLR7/8 induced IkB degradation. Unmodulated levels of intracellular readouts and PMA induced signaling were similar between SLE and HD, suggesting that 1. Signaling differences are not the result of elevated unmodulated levels of signaling and 2. Overall signaling capacity is not compromised in SLE. See FIG. 7.

SLE Donor Subgroups:

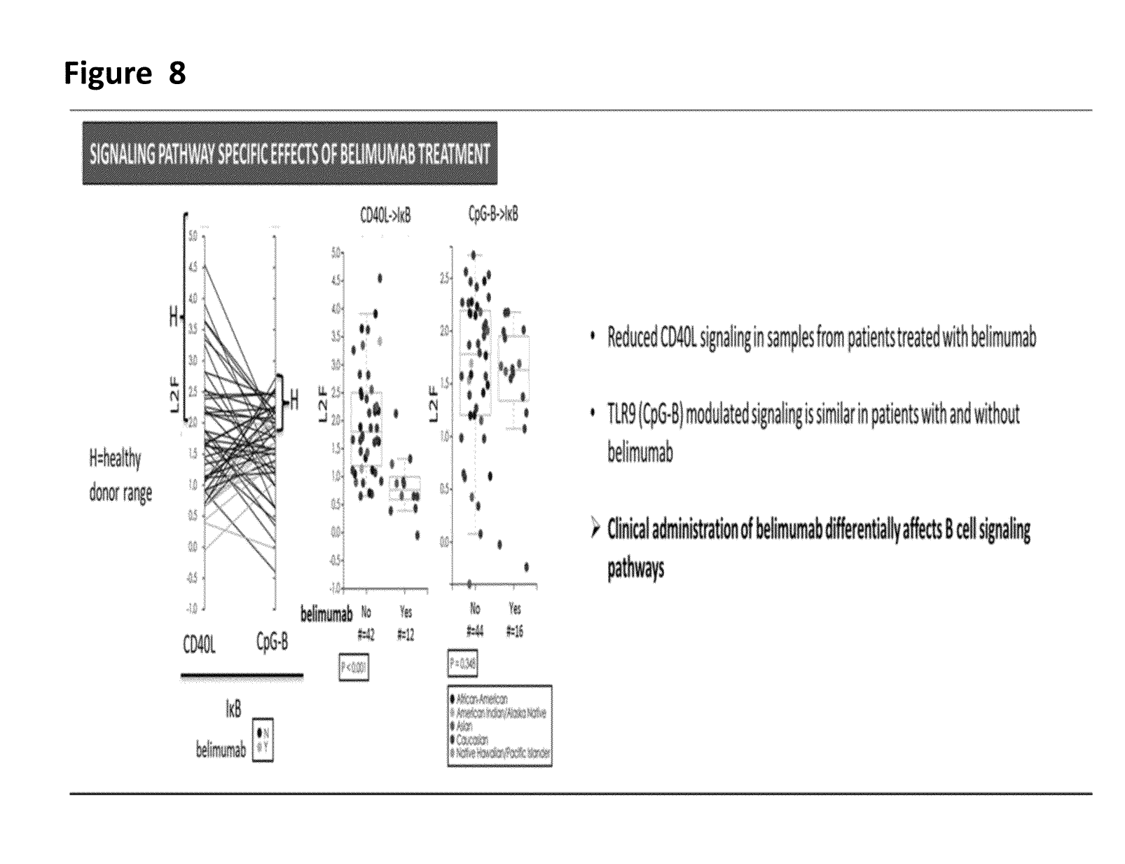

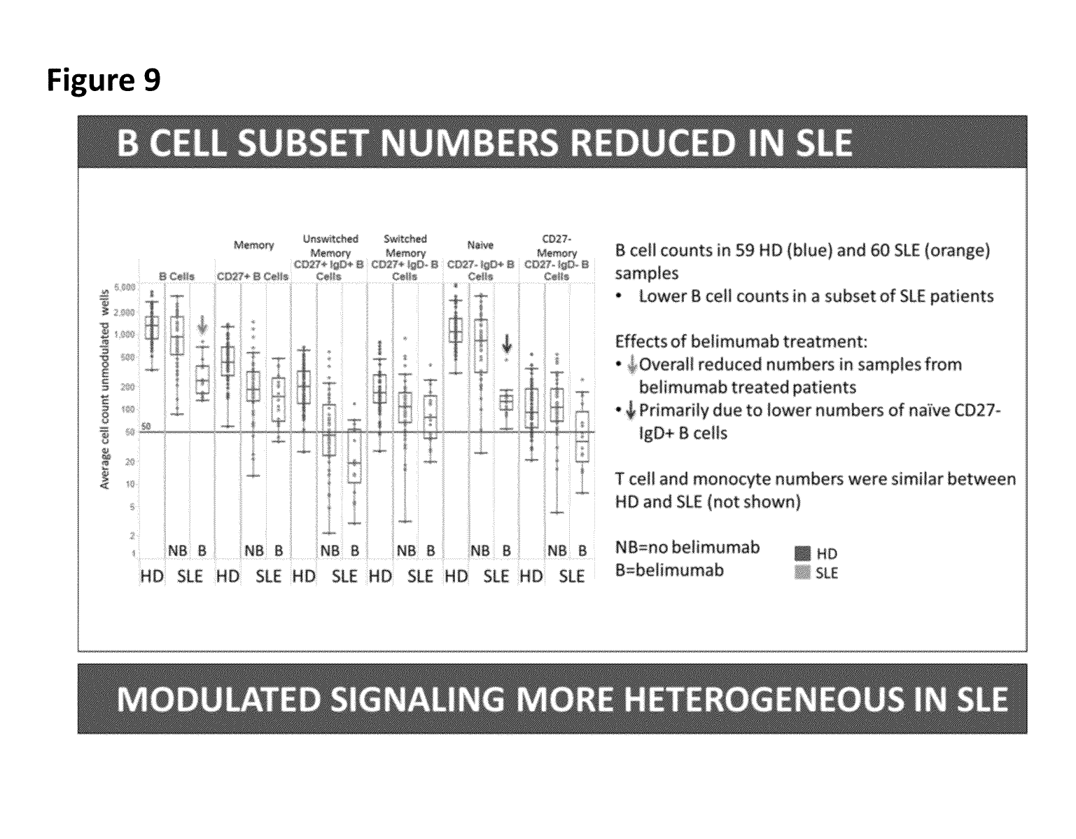

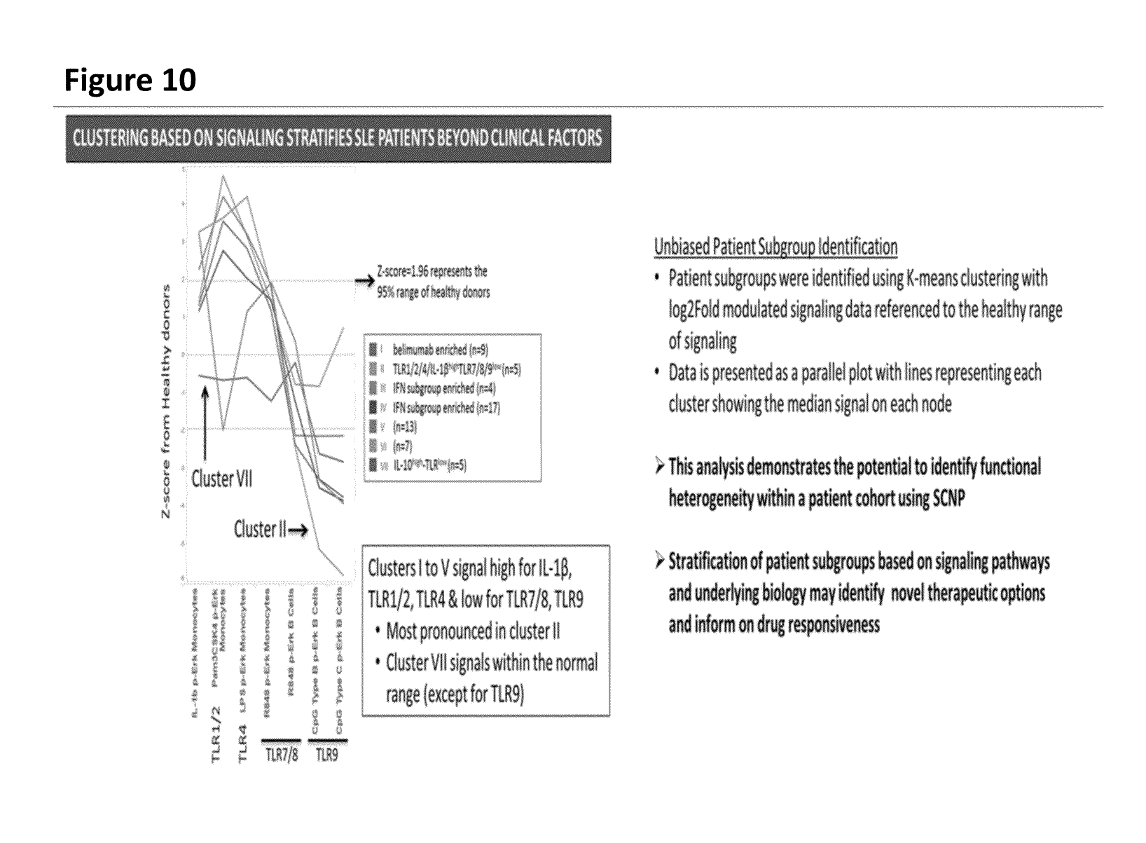

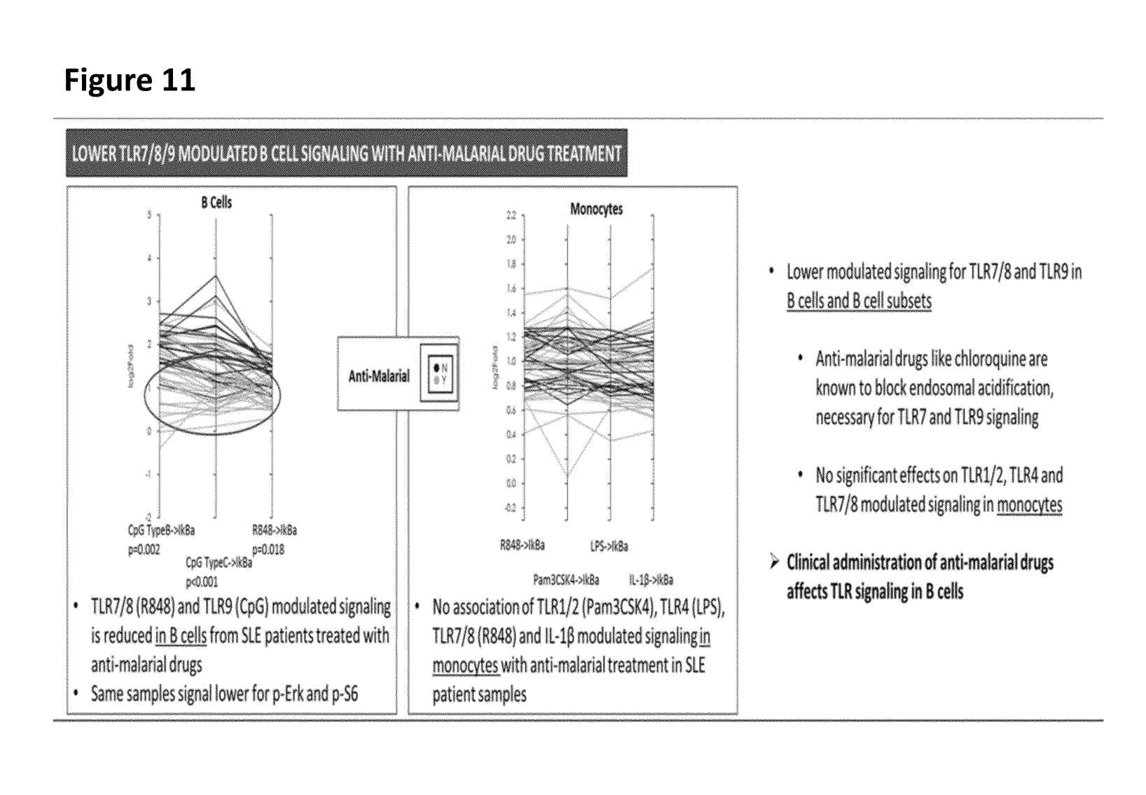

Distinct signaling profiles were identified based upon multivariate analysis of signaling within the SLE population. Not only was signaling quantitatively more broadly distributed in SLE vs HD (FIG. 6), there were also nodes in which distinct subgroups were also observed (Table 3). Associations of dysfunctional signaling with donor demographics, including belimumab treatment were found. See FIGS. 8, 9, and 10. In addition, it was found that clinical administration of anti-malarial drugs affects TLR signaling in B cells. See FIG. 11.

TABLE 3
Subgroups of SLE patients based on modulated
signaling outside the range for HD.

| | | | SLE subgroup |
|---|---|---|---|
| | | | identified with |
| | | | higher/lower |
| | Intracellular | | signaling |
| Modulator | Readout | Cell Subset | compared to HD |
| IFNα | p- | B cells, monocytes, | Lower |
| | STAT1, -3, -5 | T cell subsets | |
| IFNγ | p- | B cells, monocytes, | Higher |
| | STAT1, -3, -5 | T cell subsets | |
| IL-4 | p-STAT5 | B cell subsets | Lower |
| IL-6 | p-STAT5 | T cell subsets | Lower |
| IL-7 | p-STAT5 | B cells | Higher |
| IL-10 | p-STAT1 | Monocytes | Higher |
| IL-10 | p-STAT5 | Monocytes | Lower |
| IL-21 | p-STAT3 | B cell subsets | Lower |
| CD40L | IkB, p-AKT, | B cells | Lower |
| | p-ERK, p-S6 | | |
| Anti-IgD | p-AKT, p-S6 | B cells | Lower |
| TLR7/8, TLR9 | IkB, p-ERK | B cells | Lower |
| TLR1/2, TLR4, | IkB | Monocytes | Lower |
| TLR7/8 | | | |
| TLR1/2, TLR4, | p-ERK | Monocytes | Higher |
| TLR7/8 | | | |
| IL-1b | p-CREB, | Monocytes | Higher |
| | p-ERK, | | |
| | p-c-Jun | | |
| TLR7/8 | IkB | mDCs | Higher |

Conclusion:

These SCNP data identify both modulator-specific, disease-associated dysfunctional signaling and SLE donor subgroups based upon cell subset specific immune signaling capacity. Ongoing analyses will inform on the clinical relevance of these observations to enable functional refinement of the spectrum of SLE and identification of novel targets for therapeutic intervention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A method of determining the status of an individual diagnosed with or suspected of having SLE comprising
(i) determining the activation level of an activatable element in a cell from a sample from the individual; and
(ii) based on the level determined in (i), determining the status of the individual.

2. The method of claim 1 wherein the individual has been diagnosed with SLE and the status is current status of the disease, likelihood of a future status of the disease, or likelihood of response to treatment.

3. The method of claim 1 wherein the cell is treated with a modulator.

4. The method of claim 3 wherein the modulator is selected from the group consisting of CD40L, CpG-C, Anti-IgD, IL-1β, LPS, Pam3CSK4, PMA, R848, IFNα, IFNγ, IL-2, IL-4, IL-6, IL-7, IL-10, IL-15, IL-21, IL-27, and GMCSF.

5. The method of claim 1 wherein the activatable element is selected from the group consisting of p-Akt, p-CREB, p-Erk, IkB, p-c-Jun, p-P38, p-S6, p-Stat3, p-Stat1, p-Stat3, p-Stat5, and p-Stat6.

6. The method of claim 1 wherein the cell is a T cell, a B cell, or a monocyte, or a subset selected from the group in the TABLE.

7. The method of claim 1 wherein the activation level of two activatable elements is determined and the determination of the status comprises finding a ratio of the levels of the two activatable elements.

8. The method of claim 7 wherein the cells is treated with a modulator.

9. A method of screening an agent for potential use as a therapeutic agent in SLE, comprising exposing cells to the agent and determining the activation level of one or more activatable elements single cells, and determining the suitability of the agent for potential use as a therapeutic agent based on the activation level determined.

10. The method of claim 9 wherein the single cells are treated with a modulator.

11. The method of claim 9 wherein the modulator is selected from the group consisting of CD40L, CpG-C, Anti-IgD, IL-1β, LPS, Pam3CSK4, PMA, R848, IFNα, IFNγ, IL-2, IL-4, IL-6, IL-7, IL-10, IL-15, IL-21, IL-27, and GMCSF.

12. The method of claim 9 wherein the activatable element is selected from the group consisting of p-Akt, p-CREB, p-Erk, IkB, p-c-Jun, p-P38, p-S6, p-Stat3, p-Stat1, p-Stat3, p-Stat5, and p-Stat6.

13. The method of claim 9 wherein the cell in which the activation level of the activatable element is determined is a T cell, a B cell, or a monocyte, or a subset selected from the group in the TABLE.

14. The method of claim 9 wherein the activation level of two activatable elements is determined and the determination of the suitability of the agent comprises finding a ratio of the levels of the two activatable elements.

15. The method of claim 9 wherein the cells is treated with a modulator.
